(19) United States
(12) Patent Application Publication
(10) Pub. No.: US 2011/0236394 A1
(43) Pub. Date: Sep. 29, 2011

(54) METHODS FOR THE REGULATION OF THE PROSTAGLANDIN F SYNTHASE (PGFS) ACTIVITY OF AKR1B1 AND USES THEREOF

(75) Inventors: Michel Fortier, Quebec (CA); Pierre Chapdelaine, Saint-Romuald (CA); Eva Bresson, Quebec (CA); Eric Madore, Quebec (CA)

(73) Assignee: UNIVERSITE LAVAL, Québec, QC (CA)

(21) Appl. No.: 12/743,206

(22) PCT Filed: Nov. 14, 2008
(86) PCT No.: PCT/CA08/02012

§ 371 Date: May 9, 2011

Related U.S. Application Data
(60) Provisional application No. 60/988,220, filed on Nov. 15, 2007.

(51) Int. Cl. A61K 39/395; A61K 38/44; A61K 31/7088; A61P 9/10; A61P 15/00; A61P 3/00; A61P 11/08

(52) U.S. Cl. 424/158.1; 424/94.4; 514/44 R; 514/44 A

(57) ABSTRACT

AKR1B1 (EC 1.1.1.21) is an aldose reductase that has mainly been associated with the polyol pathway, and more recently with lipid deperoxidation. We have discovered that the primary activity of this enzyme is rather a PGFS activity, catalyzing the transformation of PGH₂ into PGF_{2α}. AKR1B1 as a therapeutic target, and method for modulating its expression and activity are provided. Methods for regulating the expression and activity of PGF_{2α} are also provided.

### FIELD OF THE INVENTION

The present invention relates to a method for modulating and monitoring PGF_{2α} levels and activity in a subject in need thereof by modulating AKR1B1 (aldose reductase) levels or its PGFS activity in the subject. AKR1B1 as a therapeutic target, and method for modulating its expression and PGFS activity are provided.

### BACKGROUND OF THE INVENTION

Prostanoids, such as prostaglandins (PGs), thromboxanes (TXs) and prostacyclin (PGI₂), are lipid compounds enzymatically derived from free fatty acids (FFAs). All prostanoids contain 20 carbon with a 5-carbon ring. Based on the initial fatty acid from which they are derived, either gamma-dihomolinolenic acid (DGLA), arachidonic acid (AA) or 5,8,11,14,17-eicosapentaenoic acid (EPA), there will be 1, 2 or 3 double bounds in the members of series 1 (ex.: PGE₁), series 2 (ex.: PGE₂) or series 3 (ex.: PGE₃) PGs respectively. The most important FFA in the western diet of both human and farm animals is AA, thus yielding the pro-inflammatory series 2 PGs. PGs are involved in a wide variety of physiological actions and processes, but are especially notorious for their involvement in pain, inflammation, thrombosis and cancer for which they have been a therapeutic target for more than a century. Altering the production of series 2 PGs or their relative proportion with omega-3 FFAs reduces insulin resistance and risks of heart disease.

The first rate-limiting step for the production of series 2 PGs is the release of AA from the cell membrane phospholipids via the phospholipase A2 (PLA2) or successive action of phospholipase C (PLC) and diglycerol lipase enzymes. AA is then converted into prostaglandin H₂ (PGH₂), the common precursor for all PGs, through the cyclooxygenase and peroxidase activities of prostaglandin H synthase (PGHS) also known as cyclooxygenase (COX). There are three COX isoforms, namely COX-1 (PGHS-1), COX-2 (PGHS-2) and COX-3 (PGHS-3).

COX-1 is a constitutively expressed enzyme localized mainly in the endoplasmic reticulum and involved in normal physiological functions, although it is however suspected of being upregulated in various carcinoma. COX-2 is predominantly localized on the nuclear envelope of the cell and its expression is induced by various growth factors, oncogenes, carcinogens and tumor-promoting phorbol esters. COX-2 has been previously associated with rheumatoid diseases, inflammation and tumorigenesis. COX-3 is a splice variant of COX-1, but its contribution in human physiological function remains to be established.

The various PGs isotypes have different and often opposed physiological effects, but share PGH₂ as their common precursor. Therefore, the various COX isoforms, which represent the current therapeutic target of pharmacological control of PG action, do not a priori exhibit a selectivity on the production of a specific PG isotype over another. The production of specific PG isotypes is rather controlled by the various terminal prostaglandin synthases, all of them utilizing PGH₂ as a substrate. Some active PGs can also be converted into another active isoform, PGD₂ and PGE₂ can be converted enzymatically into PGF_{2α} and PGD₂ spontaneously into PGJ₂.

Prostaglandin F synthase (PGFS) is a terminal prostaglandin synthase that converts PGH₂ into prostaglandin F_{2α} (PGF_{2α}). PGF_{2α} has been found to be involved in several physiological processes including, for example, contraction of smooth muscle including uterus and vascular walls, luteolysis, renal filtration and regulation of ocular pressure. It has also been associated with the initiation of menstruation and with the uterine ischemia leading to menstrual pain, while its vasoconstrictive effect is suspected of playing a role in controlling menstruation bleeding. PGF_{2α} has further been associated with premature labor, and recent observations in blood vessels, heart and nerve terminals suggested that it may contribute to complications associated with various diseases and disorders, such as diabetes, osteoporosis and menstrual disorders.

Another major PG is prostaglandin E₂ (PGE₂), produced from PGH₂ by the terminal prostaglandin synthase prostaglandin E synthase (PGES). PGE₂ often presents effects opposed to those of PGF_{2α} on many physiological functions and processes, such as luteal function and smooth muscle contraction including that of blood vessels. However, prior art studies on PGs have mostly focused on the contribution of PGE₂ to the notorious effects of PGs on pain, inflammation, and cancer, thus emphasizing the development of systemic and non-selective blockade of PG biosynthesis with Non Steroidal Anti-Inflammatory Drugs (NSAIDs) and COX inhibitors.

Positive contribution of PGs to normal physiological function has been described mainly in the female reproductive system, in which they are generally recognized as primary regulators of ovulation, uterine receptivity, implantation and parturition. In this respect, we have previously shown the importance of the balance between the relative effects of PGE₂ and PGF_{2α}. To date, most efforts have been concentrated on the identification of the PGES pathway leading to the production of PGE₂. Accordingly, the main enzyme responsible for stimulated formation of PGE₂ is microsomal PGES-1 (mPGES-1), which is considered as a major therapeutic target in different pathological conditions such as inflammation, pain, fever, anorexia, atherosclerosis, stroke and cancer.

PGF_{2α} can be synthesized from three distinct pathways (FIG. 1). The major pathway ensuring selective production of PGF_{2α} involves the reduction of PGH₂ by a PGFS, which is a 9,11-endoperoxide reductase. The other two pathways involve the reduction of PGD₂ by a 11-ketoreductase (11K-PGR) and the reduction of PGE₂ by a 9-ketoprostaglandin reductase (9K-PGR).

Until now, six enzymes having a PGFS activity have been identified. Of them, three were isolated from the cattle: lung-type PGFS (PGFS1), lung-type PGFS found in liver (PGFS2), and liver-type PGFS, also known as dihydrodiol dehydrogenase 3 (DDBX). The other three PGFS were respectively isolated from human, sheep and Trypanosoma brucei. As a group, these six enzymes belong to the AKR family, with the T. brucei enzyme belonging to the AKR5A subfamily, and the other five to the AKR1C subfamily. With the exception of the T. brucei enzyme, those enzymes also possess a 11-ketoreductase activity, thus giving them the ability to convert PGD₂ into 9α,11β-PGF_{2α}, a bioactive enantiomer of PGF_{2α}. Bovine PGFS1 and PGFS2 have a Kₘ value of 120 μM for PGD₂ and of 10 μM for PGH₂. DDBX possess Kₘ values of 10 μM for PGD₂ and of 25 μM for PGH₂. The three bovine PGFS are closely related, with PGFS1 and PGFS2 sharing 99% identity, although produced from 2 different genes. DDBX is 86% identical to both PGFS1 and PGFS2.

Previous studies on the regulation of PGFS activity in bovine endometrium led to the conclusion that none of the PGFS of the AKR1c family were responsible for PGF_{2α} production. This led to the identification of the bovine 20α-hydroxysteroid dehydrogenase (HSD) (bovine AKR1B5) as the functional PGFS responsible for PGF_{2α} production in the bovine endometrium (Madore et al., J Biol Chem 278(13); 11205-12, 2003).

Aldo-keto reductases (AKRs) are generally soluble 37 kDa monomeric NAD(P)(H)-dependent oxidoreductases capable of reducing aldehydes and ketones to yield primary and secondary alcohols. The AKR family comprises approximately 140 members sharing minimal sequence identity (less than 40% overall), divided in 15 subfamilies. AKRs having protein sequences sharing more than 60% identity are grouped into subfamilies, with mammalian AKR1 representing the largest of the 15 subfamilies.

The human PGFS AKR1C3 (EC 1.1.1.213, 1.3.1.20 and 1.1.1.62), is an aldo-keto reductase of the 1C family generally associated with a HSD activity. It has been primarily studied for its type V 17β-HSD activity, and in this respect, was found to be expressed in the human endometrium.

Aldehyde reductase (AKR1A1; EC 1.1.1.2) and aldose reductase (AKR1B1; EC 1.1.1.21) are monomeric NADPH-dependent oxidoreductases sharing 51% identity and having wide substrate specificities for carbonyl compounds.

The best known and most widely studied human AKR is the human aldose reductase AKR1B1 (E.C. 1.1.1.21), previously demonstrated to be broadly expressed throughout the body and primarily associated with the polyol pathway (reduction of glucose into sorbitol). AKR1B1 is believed to be involved in metabolic disorders such as diabetes complications and comorbidities.

More recently AKR1B1 has been presented as a detoxification enzyme protecting against toxic aldehydes derived from lipid peroxidation. (Jin Y and Penning T M, Ann Rev Pharmacol Toxicol, 2007). According to this hypothesis, a series of metabolic reactions would deplete the NAD-NADPH pool and could thus explain complications associated with metabolic disorders such as diabetes.

AKR1B1 catalyzes the reduction of various aromatic and aliphatic aldehydes, including the aldehyde form of glucose, which is reduced by AKR1B1 to its corresponding sugar alcohol, sorbitol. Sorbitol can subsequently be metabolized to fructose by sorbitol dehydrogenase. Under normal glycemic conditions, this pathway only plays a minor role in glucose metabolism in most tissues. However, in diabetic hyperglycemia, the cells undergoing insulin-independent uptake of glucose are producing significant quantities of sorbitol. This leads to an accumulation of sorbitol in the cells because of the poor penetration of sorbitol across cellular membrane and its slow metabolism by sorbitol dehydrogenase. The resulting cellular hyperosmotic stress can induce diabetic complications such as neuropathy, retinopathy, and cataracts. Further, recent studies showed that AKR1B1 also possesses a high catalytic activity towards the reduction of lipid peroxides derived from aldehydes and their glutathione conjugates, suggesting that under normal glucose conditions, AKR1B1 could therefore protects the organism against oxidative stress (Obrosova I. G. et al., Curr Vasc Pharmacol 3(3); 267-83, 2005) and electrophilic stress (Barisani D. et al., FEBS Lett 469(2-3); 208-12, 2000).

The reduction of glucose into sorbitol by AKR1B1 has thus been linked to mechanisms involved in diabetes-related disorders, such as cataracts, renal disorders, neuropathies, cardiac ischemia and cerebral ischemia. Accordingly, various AKR1B1 inhibitors have been tested in the prevention of diabetes-related disorders, in order to try to regulate AKR1B1-induced sorbitol formation. However, AKR1B1 inhibitors developed by pharmaceutical companies, such as Tolrestat™, Statil™ and Zopolrestat™, and which are administered for blocking the reduction of glucose into sorbitol in diabetic subjects having neuropathies, have often been found to be associated with undesirable hepatic side-effects (FIG. 2).

Numerous and highly complex metabolic reactions therefore appears to be involved in diabetic complications, but the mechanisms underlying AKR1B1 involvement, either from its polyol or lipid peroxidation activities, remains to be established.

NSAIDs and COX-2-specific inhibitors are widely used to treat pain, fever and inflammation. While current therapies with NSAIDs and COX inhibitors can alleviate some of the symptoms by blocking non-selectively the production of all PGs at various degrees, there is a need for a more subtle and targeted approach to treat such conditions. It is known that the production of different PG isoforms or expression of their receptors must be coordinated through crosstalk mechanisms in order to maintain homeostasis. However, under special conditions such as insulin resistance, diabetes, oxidative stress or COX inhibitor therapy, these intrinsic feedback mechanisms can become impaired or inoperative. Overproduction of PGF_{2α} relative to PGE₂ could therefore occur in such conditions, which could lead to ischemia for example. Underproduction of PGF_{2α} relative to PGE₂ could also occur, for example in the eye, which could ultimately result in an increased ocular pressure. Thus, in conditions where such intrinsic feedback mechanisms are partly or totally inoperative, new drug targets directed toward specific terminal synthases involved in the production of PGs and regulating their activity are highly desirable.

Considering the opposed effects of the various PGs on many physiological processes and functions along with the existence of regulatory crosstalk and feedback mechanisms allowing for a balanced ratio of PGF_{2α}/PGE₂, there is therefore a need for a new and more specific therapeutic target allowing fine control of specific PG isotypes production.

PGs are important regulators of female reproductive function and contribute to gynecological disorders. Menstruation depends on an equilibrium between vasoconstrictors such as PGF_{2α} and vasodilators such as PGE₂ and nitric oxide (NO). Certain disorders are known to involve a dysregulation of the balance between PGE₂ and PGF_{2α} levels. When such an unbalance implies higher levels of vasoconstrictor PGF_{2α} compared to vasodilator PGE₂, it has been observed that PGF_{2α} induces sustained muscle contractions that can lead to muscle ischemia (Lundstrom, V., Acta Obstet Gynecol Scand, 1977, 56(3); 167-72). In cases where the balance is dysregulated towards higher PGE₂ levels, abundant bleeding have been reported. Therefore, the balance of these two PGs with opposite effects is of primordial importance.

PGF_{2α} shares with TXA₂ the ability to contract smooth muscle including that of vascular walls. PGF_{2α} receptors (FP) were recently discovered in the left heart ventricle and coronaries, thus suggesting a possible implication of PGF_{2α} in cardiac ischemia. A similar pattern of expression was described in the human uterus and associated with uterine ischemia leading to menstrual pain. Moreover, following the approval of inhaled insulin for the treatment of diabetes, it was found that absorption was limited by the constriction of bronchi, an effect that could be potentiated by PGF_{2α}. Ocular pressure and renal filtration are additional mechanisms in which PGF_{2α} could play a role.

Because of its notorious role on inflammation and pain, the biosynthetic pathway leading to PGE₂ synthesis has been well studied, while the one leading to PGF_{2α} synthesis is poorly documented. The data presented herein describes for the first time the expression of AKR1B1 gene and protein and its functional association with PGF_{2α} production in the human endometrium. Also presented are methods and tools for evaluating the risk of a subject towards AKR1B1-related disorders, and for developing modulators of the PGFS activity of AKR1B1.

### BRIEF SUMMARY OF THE INVENTION

AKR1B1 (EC 1.1.1.21) is an aldose reductase that has mainly been associated with the polyol pathway, and more recently with lipid deperoxidation. We have identified that the primary activity of this enzyme is rather a PGFS activity, catalyzing the transformation of PGH₂ into PGF_{2α} (FIG. 3).

It is an aspect of the present invention to provide a method for decreasing the PGFS activity in a subject, said method comprising the step of administering an AKR1B1 inhibitor to said subject. In accordance with the present invention, the AKR1B1 inhibitor is preferably selected from the group consisting of inhibitor of AKR1B1 PGFS activity, inhibitor of AKR1B1 synthesis, inhibitor of AKR1B1 translation, inhibitor of AKR1B1 post-translational modification, regulator of AKR1B1 transit within the cytoplasm, and activator of AKR1B1 degradation; and is preferably one of a AKR1B1 siRNA and a AKR1B1 antibody. In further accordance with the present invention, the AKR1B1 inhibitor can be co-administered to the subject with at least one of a COX inhibitor, COX-2-specific inhibitor, FP receptor blocker, EP1 receptor blocker, EP3 receptor blocker, and a PGF_{2α} antagonist. In yet further accordance with the present invention, the subject is a human subject.

It is another aspect of the present invention to provide a method for decreasing the levels of PGF_{2α} in a subject, said method comprising the step of administering an AKR1B1 inhibitor to said subject. In accordance with the present invention, the AKR1B1 inhibitor is preferably selected from the group consisting of inhibitor of AKR1B1 PGFS activity, inhibitor of AKR1B1 synthesis, inhibitor of AKR1B1 translation, inhibitor of AKR1B1 post-translational modification, regulator of AKR1B1 transit within the cytoplasm, and activator of AKR1B1 degradation; and is preferably one of a AKR1B1 siRNA and a AKR1B1 antibody. In further accordance with the present invention, the AKR1B1 inhibitor can be co-administered to the subject with at least one of a COX inhibitor, COX-2-specific inhibitor, FP receptor blocker, EP1 receptor blocker, EP3 receptor blocker, and a PGF_{2α} antagonist. In yet further accordance with the present invention, the subject is a human subject.

It is another aspect of the present invention to provide a method for treating or preventing a condition associated to an increase of PGF_{2α} levels or activity in a subject, said method comprising the step of administering an AKR1B1 inhibitor to said subject. In accordance with the present invention, the condition is preferably selected from the group consisting of metabolic disorders, metabolic disorder complications, cardiac ischemia, cerebral ischemia, bronchial constriction, menstrual pain, renal dysfunction and premature labor. In accordance with the present invention, the AKR1B1 inhibitor is preferably selected from the group consisting of inhibitor of AKR1B1 PGFS activity, inhibitor of AKR1B1 synthesis, inhibitor of AKR1B1 translation, inhibitor of AKR1B1 post-translational modification, regulator of AKR1B1 transit within the cytoplasm, and activator of AKR1B1 degradation; and is preferably one of a AKR1B1 siRNA and a AKR1B1 antibody. In further accordance with the present invention, the AKR1B1 inhibitor can be co-administered to the subject with at least one of a COX inhibitor, COX-2-specific inhibitor, FP receptor blocker, EP1 receptor blocker, EP3 receptor blocker, and a PGF_{2α} antagonist. In yet further accordance with the present invention, the subject is a human subject.

It is another aspect of the present invention to provide a method for increasing the PGFS activity in a subject, said method comprising the step of administering an AKR1B1 activator to said subject. In accordance with the present invention, the AKR1B1 activator is preferably selected from the group consisting of activator of AKR1B1 synthesis, activator of AKR1B1 translation, activator of AKR1B1 binding, and inhibitor of AKR1B1 degradation, AKR1B1 gene and AKR1B1 protein; and is preferably one of a nucleic acid encoding for at least the PGFS activity portion of AKR1B1 and a polypeptide having at least the PGFS activity of AKR1B1. In further accordance with the present invention, the AKR1B1 activator can be co-administered to the subject with at least one of a COX activator, COX-2-specific activator, FP receptor activator, EP1 receptor activator, EP3 receptor activator, and a PGF_{2α} agonist. In yet further accordance with the present invention, the subject is a human subject.

It is another aspect of the present invention to provide a method for increasing the levels of PGF_{2α} in a subject, said method comprising the step of administering an AKR1B1 activator to said subject. In accordance with the present invention, the AKR1B1 activator is preferably selected from the group consisting of activator of AKR1B1 synthesis, activator of AKR1B1 translation, activator of AKR1B1 binding, and inhibitor of AKR1B1 degradation, AKR1B1 gene and AKR1B1 protein; and is preferably one of a nucleic acid encoding for at least the PGFS activity portion of AKR1B1 and a polypeptide having at least the PGFS activity of AKR1B1. In further accordance with the present invention, the AKR1B1 activator can be co-administered to the subject with at least one of a COX activator, COX-2-specific activator, FP receptor activator, EP1 receptor activator, EP3 receptor activator, and a PGF_{2α} agonist. In yet further accordance with the present invention, the subject is a human subject.

It is another aspect of the present invention to provide a method for treating or preventing a condition associated to a decrease of PGF_{2α} levels or activity in a subject, said method comprising the step of administering an AKR1B1 activator to said subject. In accordance with the present invention, the condition is preferably selected from the group consisting of hyperglycemia, inflammation and impaired renal function. In accordance with the present invention, the AKR1B1 activator is preferably selected from the group consisting of activator of AKR1B1 synthesis, activator of AKR1B1 translation, activator of AKR1B1 binding, and inhibitor of AKR1B1 degradation, AKR1B1 gene and AKR1B1 protein; and is preferably one of a nucleic acid encoding for at least the PGFS activity portion of AKR1B1 and a polypeptide having at least the PGFS activity of AKR1B1. In further accordance with the present invention, the AKR1B1 activator can be co-administered to the subject with at least one of a COX activator, COX-2-specific activator, FP receptor activator, EP1 receptor activator, EP3 receptor activator, and a PGF_{2α} agonist. In yet further accordance with the present invention, the subject is a human subject.

It is yet another aspect of the present invention to provide a method for diagnosing or predicting the occurrence of a side-effect associated with the use of a COX inhibitor in a subject, said method comprising the steps of
a) obtaining a sample from said subject following the use of said COX inhibitor by said subject;
b) measuring at least one of a parameter selected from the group consisting of AKR1B1 expression level, AKR1B1 activity level, PGF_{2α} expression level, PGF_{2α} activity level, and PGF/PGE ratio, in said sample of step a); and
c) comparing the measured parameter of step b) with a standard parameter corresponding to the same parameter measured in a normal sample, said normal sample being selected from the group consisting of a sample of the subject prior to the use of said COX inhibitor and a plurality of samples from different subjects not using said COX inhibitor;
   wherein a higher value of the measured parameter in step b) relative to the value of the standard parameter is indicative of the occurrence or of the risk of occurrence of a side-effect associated with the use of said COX inhibitor by said subject. In accordance with the present invention, the side-effect associated with the use of a COX inhibitor is selected from the group consisting of cardiovascular side-effect, cardiac ischemia, heart failure, respiratory side-effect, cerebral ischemia, polyneuropathy, vision trouble, kidney dysfunction, menstrual disorders, heartburn, nausea, vomiting, stomach pain, swelling of feet, swelling of ankle, joint pain, muscle pain, weakness, bleeding, persisting sore throat, fever, diarrhea and headache. In further accordance with the present invention, the sample is a biological fluid sample selected from the group comprising blood sample and urine sample, or a tissue sample. In yet further accordance with the present invention, the COX inhibitor is a COX-2-specific inhibitor. In yet further accordance with the present invention, the subject is a human subject.

It is yet another aspect of the present invention to provide a method for predicting or diagnosing a side-effect associated with the use of a COX inhibitor in a subject, said method comprising a) establishing a normal activity level of ARK1B1 by measuring the activity level of ARK1B1 in a normal sample, said normal sample being selected from the group consisting of a sample of the subject prior to the use of a COX inhibitor and a plurality of samples from different subjects not using said COX inhibitor; b) taking a sample from said subject following the use of said COX inhibitor; c) measuring the activity level of ARK1B1 in said sample of step b); and d) comparing the measure of the activity level of AKR1B1 of step c) with the normal activity level established at step a); wherein a higher activity level of AKR1B1 in the sample of said subject compared to the normal activity level of AKR1B1 is indicative of a risk of developing or the presence of a side-effect associated with the use of a COX inhibitor by said subject.

It is yet another aspect of the present invention to provide a method for identifying a compound for alleviating a side-effect associated with the use of a COX inhibitor, said method comprising the steps of
a) exposing a cell to a COX inhibitor, thereby producing a COX-inhibited cell;
b) measuring at least one parameter in the COX-inhibited cell of step a), wherein said parameter is selected from the group consisting of AKR1B1 expression level, AKR1B1 activity level, PGF_{2α} expression level, PGF_{2Q} activity level, and PGF/PGE ratio, thereby producing a standard parameter;
c) exposing the COX-inhibited cell of step a) to the compound, thereby producing a treated cell;
d) measuring the same parameter as in step b) in the treated cell of step c); and
e) comparing the measured parameter of step d) with the standard parameter of step b),
   wherein a lower value of the measured parameter of step d) relative to the value of the standard parameter of step b) is indicative of the compound being a compound for alleviating a side-effect associated with the use of a COX inhibitor. In accordance with the present invention, the side-effect associated with the use of the COX inhibitor is selected from the group consisting of cardiovascular side-effect, cardiac ischemia, heart failure, respiratory side-effect, cerebral ischemia, polyneuropathy, vision trouble, kidney dysfunction, menstrual disorders, heartburn, nausea, vomiting, stomach pain, swelling of feet, swelling of ankle, joint pain, muscle pain, weakness, bleeding, persisting sore throat, fever, diarrhea and headache. In further accordance with the present invention, the cell is selected from the group consisting of human endometrial epithelial cell, human endometrial stromal cell, adipocyte, endothelial cell, human umbilical vein endothelial cell, kidney cell, HEK293 cell, smooth muscle cell, myoblast, heart cell and cardiomyocyte. In further accordance with the present invention, the cell is cultured in vitro. In further accordance with the present invention, the cell is a human endometrial stromal cell deposited at the International Depository Authority of Canada under Accession number IDAC 301008-04, or a human endometrial epithelial cell deposited at the International Depository Authority of Canada under Accession number IDAC 301008-05. In yet further accordance with the present invention, the COX inhibitor is a COX-2-specific inhibitor. In yet further accordance with the present invention, the subject is a human subject.

It is yet another aspect of the present invention to provide a method for identifying a compound for alleviating a side-effect associated with the use of a COX inhibitor, said method comprising the step of a) providing the COX inhibitor to a cell system; b) providing the compound to the cell system, thereby producing a treated cell system; c) measuring at least one of the expression level and activity level of at least one of AKR1B1 and PGF_{2α} in the treated cell system of step b); and d) comparing the at least one of the expression level and activity level of at least one of AKR1B1 and PGF_{2α} in the treated cell system of step c) with at least one of the expression level and activity level of at least one of AKR1B1 and PGF_{2α} in a non-treated cell system; wherein a lowering of at least one of the expression level or the activity level of at least one of AKR1B1 and PGF_{2α} in the treated cell system with the at least one of the expression level and activity level of at least one of AKR1B1 and PGF_{2α} in the non-treated cell system is indicative of the compound being a compound for alleviating a side-effect associated with the use of the COX inhibitor.

It is yet another aspect of the present invention to provide the use of a PGF/PGE ratio for the identification of a compound alleviating a side-effect associated with the use of a COX inhibitor, wherein said compound induces a decrease in the value of PGF/PGE ratio in a cell treated with said COX inhibitor. In accordance with the present invention, the side-effect associated with the use of the COX inhibitor is selected from the group consisting of cardiovascular side-effect, cardiac ischemia, heart failure, respiratory side-effect, cerebral ischemia, polyneuropathy, vision trouble, kidney dysfunction, menstrual disorders, heartburn, nausea, vomiting, stomach pain, swelling of feet, swelling of ankle, joint pain, muscle pain, weakness, bleeding, persisting sore throat, fever, diarrhea and headache. In yet further accordance with the present invention, the COX inhibitor is a COX-2-specific inhibitor. In yet further accordance with the present invention, the subject is a human subject.

It is yet another aspect of the present invention to provide a human endometrial stromal cell line deposited at the International Depository Authority of Canada under Accession number IDAC 301008-04. It is another aspect of the present invention to provide a human endometrial epithelial cell line deposited at the International Depository Authority of Canada under Accession number IDAC 301008-05. It is a further aspect of the present invention to provide the use of the human endometrial stromal cell line having the Accession number IDAC 301008-04, and/or the human endometrial epithelial cell line having the accession number IDAC 301008-05 for the identification of a compound for alleviating a side-effect associated with the use of a COX inhibitor.

It is yet another aspect of the present invention to provide a method for alleviating a side-effect associated with the use of a COX inhibitor in a subject, said method comprising the step of administrating a PGF_{2α} inhibitor to said subject. In accordance with the present invention, the PGF_{2α} inhibitor is selected from the group consisting of inhibitor of PGF_{2α} synthesis, inhibitor of AKR1B1 PGFS activity, inhibitor of PGF_{2α} binding, FP receptor blocker, EP1 receptor blocker, EP3 receptor blocker, and PGF_{2α} antagonist. In further accordance with the present invention, the side-effect associated with the use of the COX inhibitor is selected from the group consisting of cardiovascular side-effect, cardiac ischemia, heart failure, respiratory side-effect, cerebral ischemia, polyneuropathy, vision trouble, kidney dysfunction, menstrual disorders, heartburn, nausea, vomiting, stomach pain, swelling of feet, swelling of ankle, joint pain, muscle pain, weakness, bleeding, persisting sore throat, fever, diarrhea and headache. In yet further accordance with the present invention, the subject is a human subject, and in yet further accordance the subject has diabetes or insulin resistance.

It is yet another aspect of the present invention to provide a method for diagnosing or predicting at least one of a metabolic disorder, metabolic disorder complication, and a cardiac problem in a subject, said method comprising the steps of
a) obtaining a sample from a subject;
b) measuring the concentration of a PGF variant and a PGE variant in the sample of step a);
c) determining the PGF/PGE ratio; and
d) comparing the PGF/PGE ratio of step c) with a standard PGF/PGE ratio reflective of the absence of a metabolic disorder, metabolic disorder complications or a cardiac risk, said standard PGF/PGE ratio being determined, previously or concurrently, from the measurement of the concentration of a standard PGF variant and a standard PGE variant in a plurality of samples from a plurality of subjects not affected by said metabolic disorder, metabolic disorder complication or cardiac problem;
   wherein a higher value of the determined PGF/PGE ratio of step c) relative to the standard PGF/PGE ratio is indicative of the presence or the risk of developing at least one of the metabolic disorder, metabolic disorder complication and cardiac problem by said subject. In accordance with the present invention, the PGF variant is PGFM and the PGE variant is PGEM. In yet further accordance with the present invention, the measuring of the concentration of PGFM and PGEM in step b) is performed by anti-PFGM and anti-PGEM antibodies. In further accordance with the present invention, the PGF variant is PGF_{2α} and the PGE variant is PGE₂. In yet further accordance with the present invention, the measuring of the concentration of PGF_{2α} and PGE₂ in step b) is performed by anti-PGF_{2α} and anti-PGE₂ antibodies. In yet further accordance with the present invention, the sample is a biological fluid sample selected from the group comprising blood sample and urine sample, or a tissue sample. In yet further accordance with the present invention, the metabolic disorder is selected from the group consisting of obesity, type 2 diabetes, and insulin resistance. In yet further accordance with the present invention, the metabolic disorder complication is selected from the group consisting of osteoporosis, menstrual disorders, neuropathy, retinopathy, renal dysfunction and cataracts. In yet further accordance with the present invention, the cardiac problem is selected from the group consisting of cardiac ischemia and heart failure. In yet further accordance with the present invention, the measuring of the concentration of PGF variant and PGE variant in step b) is performed by immunoassay. In yet further accordance with the present invention, the immunoassay is an ELISA. In yet further accordance with the present invention, the subject is a human subject.

It is yet another aspect of the present invention to provide a method for predicting at least one of a metabolic disorder and a cardiac problem, said method comprising the steps of a) taking a sample from a subject; b) measuring the concentration of PGFM and PGEM in the sample of step a); c) determining the PGFM/PGEM ratio; and d) comparing the PGFM/PGEM ratio of step c) with a normal PGFM/PGEM ratio value reflective of the absence of a metabolic disorder and a cardiac risk; wherein a higher PGFM/PGEM ratio determined from the sample of said subject compared to the normal PGFM/PGEM ratio is indicative of a risk of developing at least one of a metabolic disorder and a cardiac problem by said subject.

It is yet another aspect of the present invention to provide an immunoassay kit for determining a PGFM/PGEM ratio, said immunoassay kit comprising a container with anti-PGFM antibodies and a container with anti-PGEM antibodies.

It is yet another aspect of the present invention to provide a use of a PGF/PGE ratio for diagnosing or predicting at least one of a metabolic disorder and a cardiac problem in a subject, wherein said subject has a higher value of PGF/PGE ratio than a standard PGF/PGE ratio determined from a plurality of subjects not affected by said metabolic disorder or cardiac problem.

### BRIEF DESCRIPTION OF THE DRAWINGS

Having thus generally described the aspects of the invention, reference will now be made to the accompanying drawings, showing by way of illustration, preferred embodiments thereof, and in which:

FIG. 1 illustrates the prostaglandin biosynthesis pathways, with cPLA2 releasing arachidonic acid (AA) from membrane phospholipids and PGH synthases (PGHSs, also known as COX enzymes (COX-1 and COX-2)) converting it to PGH₂. PGH₂ is converted into one of the active PG by specific terminal synthases such as PGE synthase (PGES, such as mPGES-1, mPGES-2, cPGES), PGF synthase (PGFS, such as AKR1B1, AKR1C3), prostacyclin synthase (PGIS) and thromboxane synthase (TBXAS1). PGE₂ and PGF_{2α} can be inactivated respectively into PGEM and PGFM by prostaglandin dehydrogenase (PGDH) before being cleared in urine.

FIG. 2 illustrates a summary of clinical trials outcome following the use of inhibitors developed against the aldose reductase activity of AKR1B1 (polyol pathway). Since these inhibitors were developed and tested against a secondary activity of AKR1B1 rather than against its primary, PGFS activity, no prediction can be made regarding their potential efficiency for blocking the PGFS activity of AKR1B1 without producing the documented side effects.

FIG. 3 illustrates PGF_{2α} biosynthesis as the primary activity of AKR1B1.

FIG. 4 illustrates the expression of COX-1, COX-2, AKR1B1 and AKR1C3 mRNA during the menstrual cycle as measured by competitive RT-PCR, and comprises FIG. 4A (COX-1), 4B (COX-2), 4C (AKR1B1) and 4D (AKR1C3). Results are expressed in competitor equivalent for each enzyme. Each point represents one sample. Bars represent the mean for each group. n=8 for the first 4 groups and n=7 for the last two (some points may overlap). The same extracts were used for all enzymes tested.

FIG. 5 illustrates the effect of interleukin-1β (IL-1β) on COX-1, COX-2 and AKR1B1 expression and PGF_{2α} production in human endometrial epithelial (HIEEC) and stromal (HIESC) cells. Cells were treated with increasing doses of IL-1β and PGF_{2α} biosynthetic enzymes expression and production were measured. The increase in PGF_{2α} production was correlated with a significant increase in expression of AKR1B1, COX-1 and COX-2 in HIEEC, and only of COX-2 in HIESC. In the COX-1-expressing HIEEC, COX-2-specific inhibitor NS-398 did not completely inhibited PGF_{2α} production, thus suggesting a cooperation between AKR1B1 and COX-1 for PGF_{2α} production in epithelial cells.

FIG. 6 comprises FIGS. 6A, 6B and 6C. FIG. 6A illustrates a western blot analysis of COX-2 in wild-type HIESC2 cells in relation with AKR1B1 in the presence and absence of IL-1β and AKR1B1 siRNAs. FIG. 6B illustrates the PGF_{2α} production as measured following treatment in absence or presence of IL-1β for 24 hours. FIG. 6C illustrates the expression of the alternate PGFS AKR1C3 as studied by Western analysis in wild type epithelial HIEEC-22 and stromal HIESC-2 cell lines and following transfection of HIESC-2 with a plasmid containing the AKR1C3 gene.

FIG. 7 illustrates the feedback loop regulating PGF_{2α} and PGE₂ production. Inhibition of PGF_{2α} release through downregulation of AKR1B1 or blockade of the FP receptor exerted a negative action on PGE₂ production. Blockade of the FP receptor prevented the release of early growth response factor 1 (EGR-1) to induce PGE₂ production.

FIG. 8 illustrates the demonstration of AKR1B1 as a functional PGFS in the human endometrium, and comprises FIGS. 8A, 8B, 8C and 8D. FIG. 8A illustrates the PGFS activity of purified recombinant AKR1B1 (top: conversion of PGH₂ into PGF_{2α} (TLC); bottom: metabolism of PGH₂ in presence of NADPH); FIG. 8B illustrates the increased production of PGF_{2α} in human endometrial cells transfected to overexpress AKR1B1; FIG. 8C illustrates the selective gene inactivation of PGFS (AKR1B1) mRNA and protein by specific siRNA transfected into human endometrial cells; and FIG. 8D illustrates the inhibition of PGF_{2α} production following AKR1B1 knockdown.

FIG. 9 comprises FIG. 9A and FIG. 9B, with FIG. 9A illustrating the effect of glucose on PGF_{2α} in endometrial cells; and FIG. 9B illustrating the effect of glucose on PGE₂ production in endometrial cells. Increasing doses of glucose are reflective of the high physiological (diabetes) range. When endometrial stromal cells were stimulated with IL-1β to increase PG production, glucose inhibited PGF_{2α} (FIG. 9A) and stimulated PGE₂ (FIG. 9B) production in a dose dependent manner. This suggests that aberrant glucose concentrations encountered in diabetes are able to alter the balance in the PGF_{2α}/PGE₂ ratio. This was observed in absence of alteration in AKR1B1 or mPGES-1 expression.

FIG. 10 comprises FIG. 10A and FIG. 10B, with FIG. 10A illustrating the effect of acetylsalicylic acid (ASA) on PGF_{2α} production; and FIG. 10B illustrating the effect of ASA on AKR1B1 expression. This shows that ASA targets PGF_{2α} production at two distinct levels, COX and AKR1B1, thus making it a highly effective mean to reduce PGF_{2α} production mutually supporting their respective effects on cardiac ischemia.

FIG. 11 illustrates the effect of PG receptor antagonists (FPA: FP receptor antagonist; EPA: EP receptor antagonist) on PGE₂ production in endometrial stromal cells (HIESC). Cells were treated with IL-1β to stimulate PG production in presence and absence of FPA (AL 8810) or EPA (AH 6809), and PG production was measured. Inhibition of the FP receptor, but not of the EP receptor, reduced PGE₂ production, showing that PGF_{2α} is able to regulate PGE₂ production.

FIG. 12 includes FIGS. 12A and 12B, with FIG. 12A illustrating the regulation of the PGF_{2α}/PGE₂ ratio in endometrial cells under normal conditions, where the production of PGE₂ primarily driven by mPGES-1 strictly associated with COX-2 and the production of the opposing PGF_{2α} by AKR1B1 associated with either COX-2 or COX-1, whereas a feedback loop originating from an increased PGF_{2α} production stimulates PGE₂ production through the FP receptor in order to maintain a constant PGF_{2α}/PGE₂ ratio; and FIG. 12B illustrating the effect of the blockade of COX-2 by a selective COX inhibitor, rendering the COX-2 dependent feedback mechanism inoperative and leading to the exclusive production of PGF_{2α}, thus driving up the PGF_{2α}/PGE₂ ratio and favoring pro-ischemic conditions.

FIG. 13 illustrates the proposed screening test measuring the PGFM/PGEM ratio in relation with a dysregulation of AKR1B1. In turn, aberrant PGF_{2α} production, compensated or not with increased PGE₂ will result in altered levels of the corresponding circulating and urinary PG metabolites. Thus the proposed screening test measuring the PGFM/PGEM ratio in relation with dysregulation of AKR1B1 to follow up on the effect of chronic use of COX inhibitors or as a biological marker of risks of cardiovascular diseases.

FIG. 14 illustrates the immunohistochemical analysis of AKR1B1 protein expression in human endometrium during the menstrual cycle, and comprises FIG. 14A (control, proliferative phase), 14B (AKR1B1, proliferative phase), 14C (control, secretory phase) and 14D (AKR1B1, secretory phase). Control was performed with a pre-immune serum. AKR1B1 serum was used at a dilution of 1:750. Abundant expression of AKR1B1 in luminal and glandular epithelium and in stroma is observed during the secretory phase.

FIG. 15 illustrates the effect of tumor necrosis factor α (TNFα) on COX-1, COX-2 and AKR1B1 expression and PGF_{2α} production in human endometrial epithelial (HIEEC) and stromal (HIESC) cells. Cells were treated with increasing doses of TNFα and PGF_{2α} biosynthetic enzymes expression and production were measured. The increase in PGF_{2α} production was correlated with a significant increase in expression of AKR1B1, COX-1 and COX-2.

FIG. 16 illustrates the human endometrial cell lines HIESC-2 (expressing COX-2) and HIEEC-22 (expressing both COX-1 and COX-2) as models for testing the effect of COX inhibitors and NSAIDs on different PG isoforms in an integrated manner The characteristic inhibition pattern of individual COX inhibitors on endometrial cells, particularly their relative effect on the PGF_{2α}/PGE₂ ratio, reflected the relative cardiovascular safety NSAIDs.

FIG. 17 illustrates the effect of Aspirin™ and naproxen on the production of PGF_{2α} by HIEEC cells grown to confluency and treated with IL-1β, as measured by enzyme-linked immunosorbent assay (ELISA), with IC₅₀ Aspirin: 1.941e-006 (7.950e-007 to 4.741e-006) and IC₅₀ naproxen: 4.621e-009 (9.311e-010 to 2.293e-008).

FIG. 18 comprises FIGS. 18A and 18B, with FIG. 18A illustrating the effect of various doses of naproxen on the production of PGF_{2α} and PGE₂ by stromal HIESC cells stimulated by IL-1β as measured by ELISA, with IC₅₀ PGE₂: 6.259e-008 (2.906e-008 to 1.348e-007) and IC₅₀ PGF_{2α}: 4.383e-008 (1.668e-008 to 1.152e-007); and FIG. 18B illustrating the effect of various doses of naproxen on the production of PGF_{2α} and PGE₂ by epithelial HIEEC cells stimulated by IL-1β as measured by ELISA, with IC₅₀ PGE₂: 1.419e-007 (3.163e-008 to 6.366e-007) and IC₅₀ PGF_{2α}: 4.621e-009 (9.311e-010 to 2.293e-008).

FIG. 19 comprises FIGS. 19A, 19B, 19C and 19D, and illustrates the effect of IL-1β and TNF-α on the production of PGF_{2α} (A), COX-2 (B) and AKR1B1 (C) by human primary cardiomyocytes, as measured by ELISA and Western blot. FIG. 19D illustrates β-actin levels of the three cells groups.

FIG. 20 comprises FIGS. 20A and 20B, with FIG. 20A illustrating the effect of IL-1β on the production of PGF_{2α} by primary human umbilical artery smooth muscle cells (HUASMC), as measured by ELISA; and FIG. 20B illustrating the effect of IL-1β on the protein expression of COX-2 and AKR1B1 in primary HUASMC.

FIG. 21 comprises FIGS. 21A and 21B, with FIG. 21A illustrating the effect of IL-1β on the production of PGF_{2α} by primary human umbilical vein smooth muscle cells (HUVSMC), as measured by ELISA; and FIG. 21B illustrating the effect of IL-1β on the protein expression of COX-2 and AKR1B1 in primary HUVSMC.

FIG. 22 comprises FIGS. 22A and 22B, with FIG. 22A illustrating the effect of IL-1β and TNF-α on the production of PGF_{2α} by primary human umbilical vein endothelial cells (HUVEC), as measured by ELISA; and FIG. 22B illustrating the effect of IL-1β and TNF-α on the protein expression of COX-2 and AKR1B1 in primary HUVEC.

FIG. 23 comprises FIGS. 23A, 23B and 23C, and illustrates the effect of various concentrations of rofecoxib (Vioxx™) on the production of PGE₂ (A) and PGF_{2α} (B) by HIEEC cells treated with IL-1β, as measured by ELISA; with FIG. 23C illustrating the greater efficiency of rofecoxib in inhibiting PGE₂ than PGF_{2α} in HIEEC cells stimulated with IL-1β, with IC₅₀ PGE₂: 5.174e-008 (3.256e-008 to 8.223e-008) and IC₅₀ PGF_{2α}: 2.007e-007 (1.066e-007 to 3.777e-007).

FIG. 24 comprises FIGS. 24A, 24B, 24C and 24D, and illustrates the effect of the FP receptor inhibitor AL8810 (A, B) and of the EP receptor inhibitor AH6809 (C, D) on the production of PGE₂ by IL-1β-stimulated epithelial HIEEC (A, C) and IL-1β-stimulated stromal HIESC (B, D) cells.

### DETAILED DESCRIPTION

The present invention will be more readily understood by referring to the following examples which are given to illustrate the invention rather than to limit its scope.

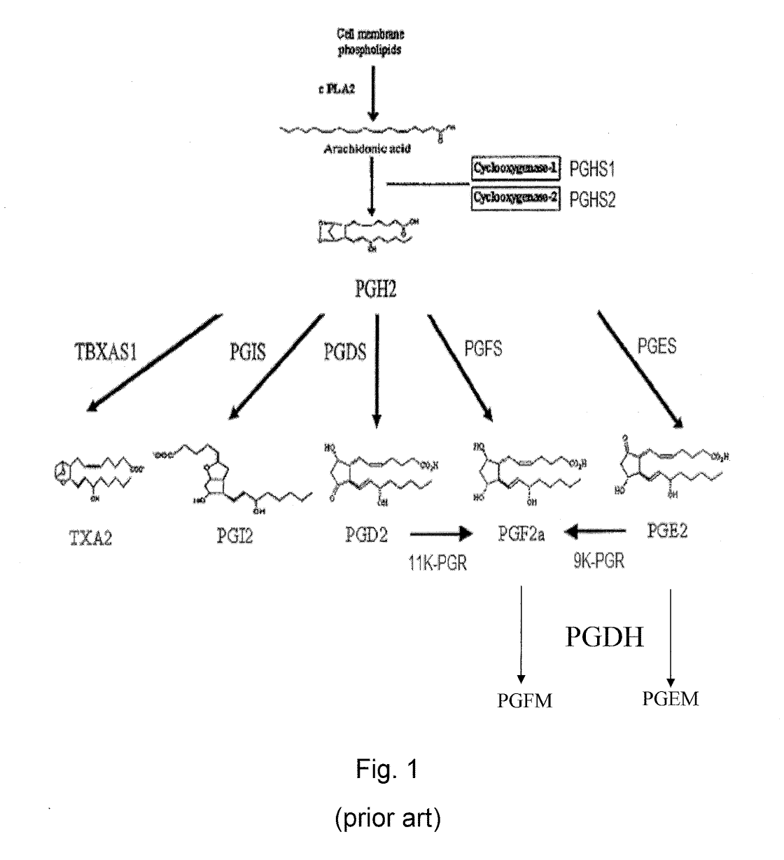

All series 2 PGs originate from the same precursor, PGH₂, which is synthesized from AA by the COXs enzymes. Specific terminal prostaglandin synthases can use this common substrate to produce specific PGs, with notably PGES catalyzing the synthesis of PGE₂, and PGFS catalyzing the formation of PGF_{2α} (FIG. 1). Series 1 and 3 PGs originates from PGH₁ and PGH₃, which are respectively converted into PGF_{1α} and PGF_{3α} by PGFS.

We evaluated a potential link between the two main COX isoforms, COX-1 and COX-2, and the various PG synthases with stimulators, inhibitors and knock-down experiments using siRNA. We confirmed the association between COX-2, mPGES-1 and PGE₂. We also found an association between AKR1B1, both COX-1 and COX-2 and PGF_{2α}, while a knock-down of AKR1B1 led to reduced levels of PGF_{2α} but also of PGE₂.

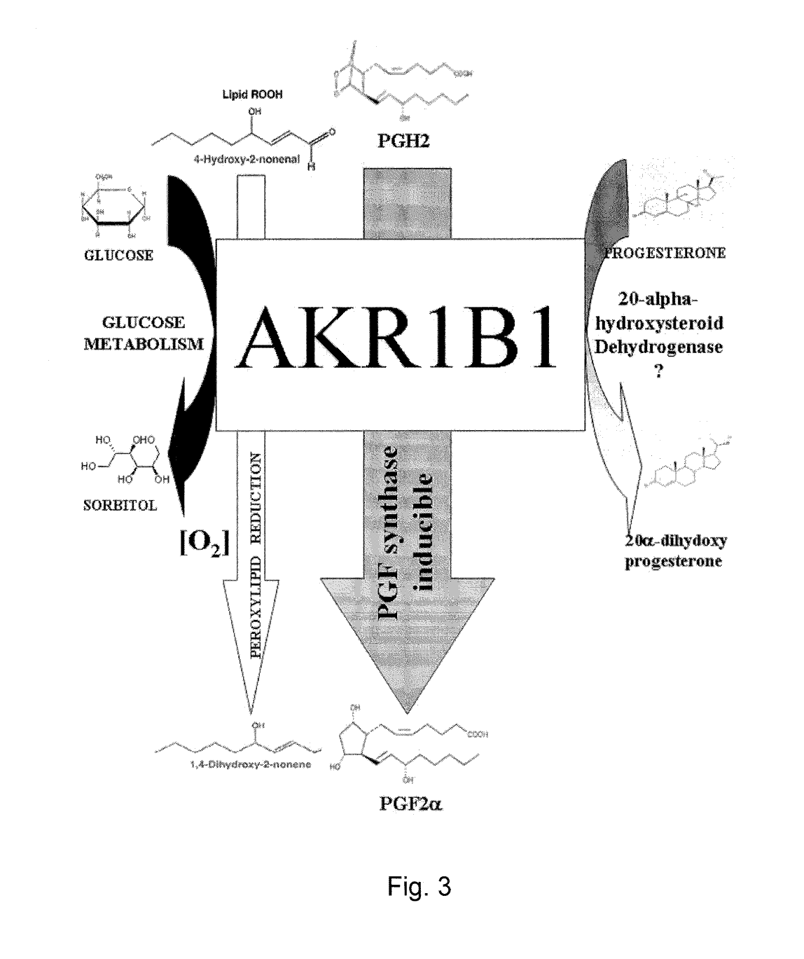

AKR1B1 was isolated and characterized with respect to transformation of glucose into sorbitol. However, this action occurs only in hyperglycemia, such as in diabetic subjects, since the glucose levels associated with normal glycemia conditions are not high enough to constitute a substrate for AKR1B1. In addition of catalyzing the formation of sorbitol from glucose, AKR1B1 also has a detoxificating action on peroxidized lipids, as reported by Srivastava (Srivastava et al., Endocr Rev 26(3); 380-92, 2005). At this point, it is worth noting that PGH₂ is a peroxidized lipid. While Srivastava mentions that AKR1B1 exerts its detoxificating action by destroying peroxidized lipids, we rather propose that AKR1B1 has a constitutive physiological role within the organism, converting PGH₂ into PGF_{2α}, a biological molecule acting through specific receptors (FIG. 3).

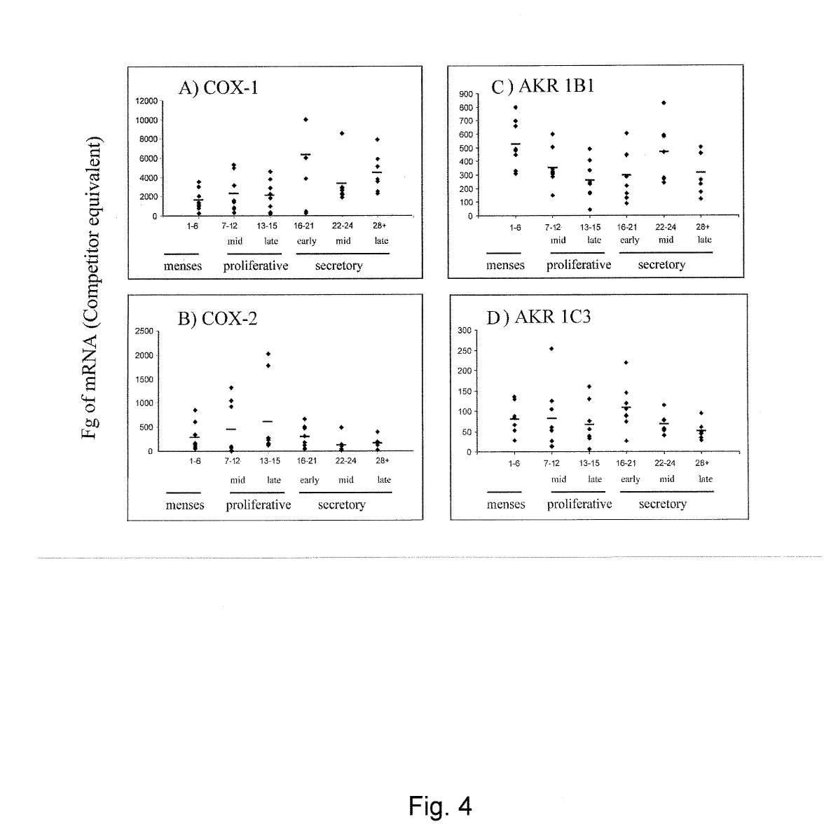

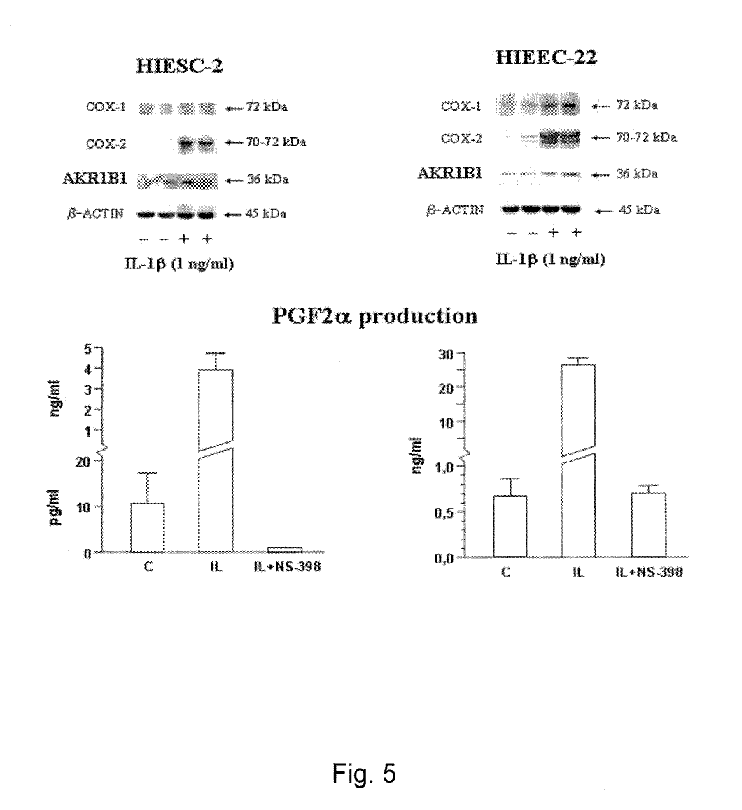

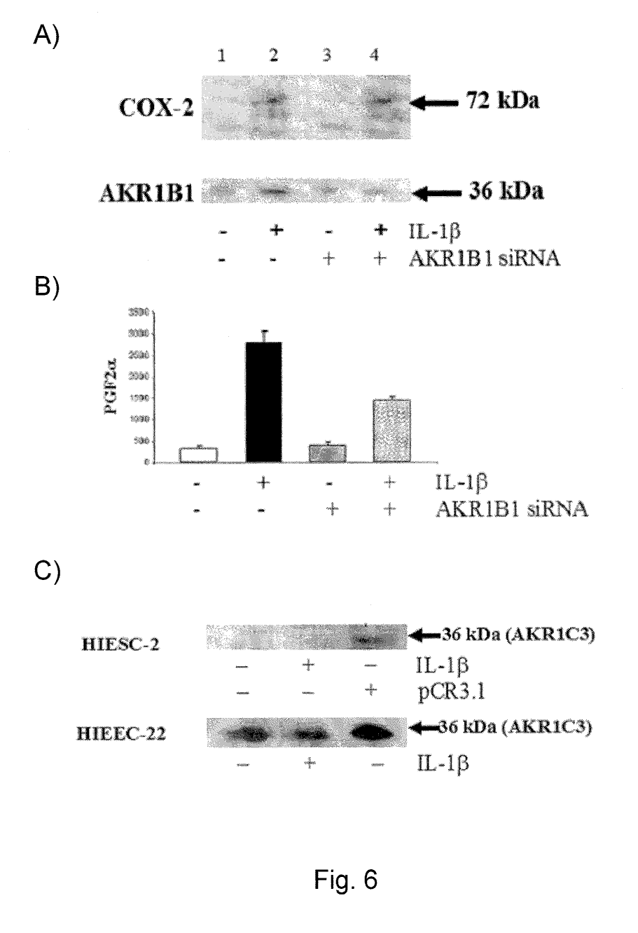

The newly identified PGFS AKR1B1, along with the known PGFS AKR1C3, are both present in the endometrium throughout the menstrual cycle (FIG. 4), with AKR1B1 being expressed in both stromal and glandular epithelial cells (FIG. 5), whereas AKR1C3 was only found in epithelial cells (FIG. 6C) and blood vessels. Because epithelial and stromal cells present similar patterns of regulation of PGF_{2α} production in response to IL-1β, and since only the AKR1B1 pathway is functional in stromal cells, we considered this AKR1B1 pathway as the preferred pathway responsible for PGF_{2α} production in the endometrium. The contribution of AKR1B1 to PGs production has never been anticipated before our studies in the endometrium.

AKR1B1 has been traditionally associated with reduction of glucose and diabetes-induced oxidative stress. Accordingly, AKR1B3 (the mouse aldose reductase now referred to as mouse AKR1B1) knockout mice have been used to study the pathogenesis of various diseases associated with diabetes mellitus, such as cataract, retinopathy, neuropathy and nephropathy. Reduced pathological responses were observed in these animals despite reduced intrinsic expression of aldose reductase in mice compared with humans. Interestingly, transgenic mice overexpressing human AKR1B1 have been found to be more prone to myocardial ischemic injury whereas knockout mice appeared to be protected against cerebral ischemic injury (Lo A C et al, J Cereb Blood Flow Metab. 2007 August; 27(8):1496-509), but the relationship between AKR1B1 and PGF_{2α} was never established or even suggested.

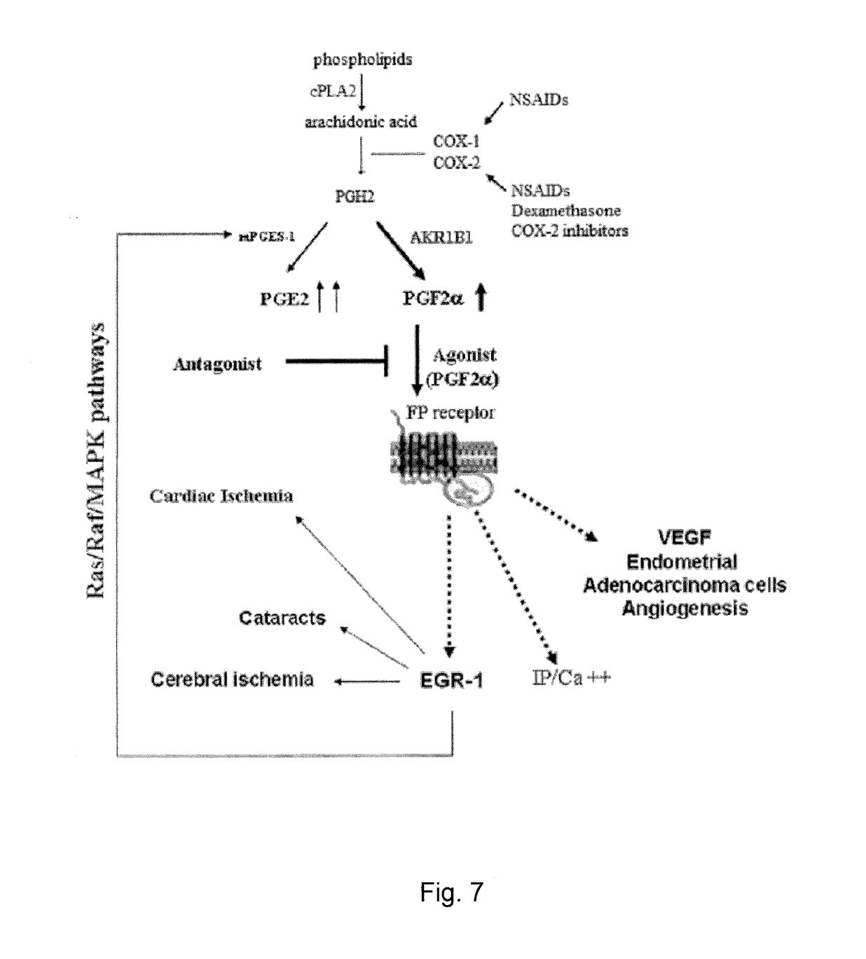

With regards to the ratio between PGF_{2α} and PGE₂, one of the principal mechanism for preserving this ratio is the existence of a retro-feedback mechanism inducing an increase of PGE₂ production by mPGES-1 via the early growth response factor 1 (EGR-1) transcription factor. This allows PGF_{2α} produced by AKR1B1 to bind to its own membrane receptor (FP) and stimulate the expression of mPGES-1 (FIG. 7). Therefore, an excess of PGF_{2α} for example would induce an increase in mPGES-1 enzymatic activity, thus increasing the synthesis of PGE₂ and restoring the balance between PGF_{2α} and PGE₂ levels. In this respect, we observed that when AKR1B1 was knocked-down using siRNAs, mPGES-1 activity was also reduced by the siRNAs. We therefore proposed that increased AKR1B1 activity releasing an excess PGF_{2α} drives a compensatory mechanism through COX-2 and mPGES-1 that leads to an increased PGE₂ production.

These observations showed that AKR1B1 could be involved in the regulation of vascular tone under conditions where glucose metabolism is not involved. However, in presence of high glucose levels associated with diabetes, glucose becomes available as a substrate for AKR1B1 and competition among substrates may explain the development of vascular and neurological complications.

In the human endometrium, it has been previously reported that production of PGF_{2α} is higher in late secretory and menstrual phases of the menstrual cycle. We have shown that AKR1B1 gene and protein levels increased significantly during the corresponding periods of menstrual cycle, whereas AKR1C3 does not vary (FIG. 4). Since we and others have previously shown that human endometrial stromal cells produce PGF_{2α}, and since AKR1C3, the only documented PGFS in human, is absent from stromal cells, an alternate enzyme needs to be responsible for the high levels of PGF_{2α} produced by these cells. However, in epithelial cells, both enzymes are expressed, despite an absence of regulation of AKR1C3 during the cycle in vivo, or in vitro by IL-1p, as opposed to AKR1B1.

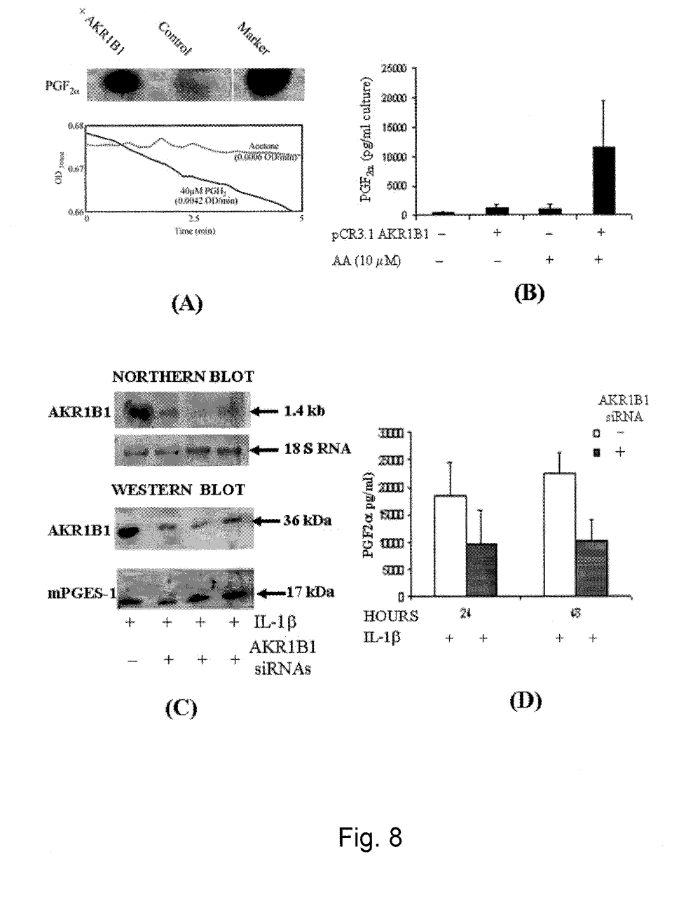

We have established that human AKR1B1 is capable of metabolizing PGH₂ and synthesizing PGF_{2α} with a high efficiency (FIG. 8). In fact, the PGFS activity of AKR1B1 uses PGH₂ at concentrations well within the physiological range, whereas the high glucose levels necessary for allowing the aldose reductase activity of AKR1B1 are generally only encountered under exceptional or pathological conditions. We found that transfection of AKR1B1 in epithelial or stromal cells increased the production of PGF_{2α} by these cells, whereas knocking down AKR1B1 expression with specific siRNA reduced the production of PGF_{2α} by these cells. By considering the minimal distribution and expression levels of AKR1C3 in the human endometrium, our results showed that AKR1B1 is the main functional PGFS responsible for most of the PGF_{2α} production in human endometrium, while the contribution of AKR1C3 is likely negligible and accessory.

Because of their association with inflammation and other pathological conditions, PGs as a whole are often considered as disorder-related molecules. Moreover, since successful clinical management of PGs is possible with NSAIDs, PGs are generally perceived as a single, unique factor. Accordingly, only two limiting steps are currently acknowledged in the synthesis of PGs: the release of AA from membrane phospholipids by phospholipases, and the generation of the intermediate PG metabolite PGH₂ by COXs. However, these steps lead to the synthesis of a common precursor for several bioactive mediators, and not a priori directly to a specific PG isotype. PGs induce a wide variety of responses mediated by receptors, which are distinct for each PG isotype and are using various second messenger systems. For example, TXA₂ and PGI₂ exert opposing effects on coagulation and vascular tone to regulate hemostasis, while in the reproductive system, opposite actions are observed for PGF_{2α} and PGE₂.

In subjects affected with insulin resistance, insulin secretion is increased to maintain normal glucose levels. However, in these subjects, only one component of the insulin receptor is desensitized, corresponding to the PI3K pathway, whereas the other MAPK pathway remains intact. Therefore, higher insulin levels in insulin-resistant subjects are required to maintain normal glucose levels (silent condition), but the expression of insulin-responsive genes, including AKR1B1, are aberrantly expressed in response to these higher insulin levels.

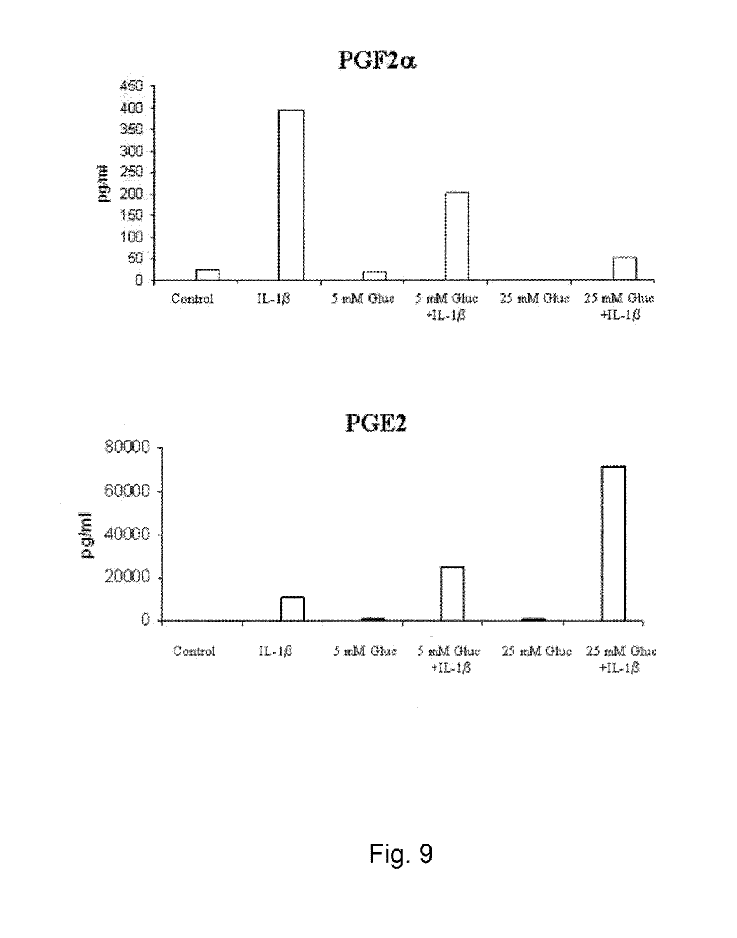

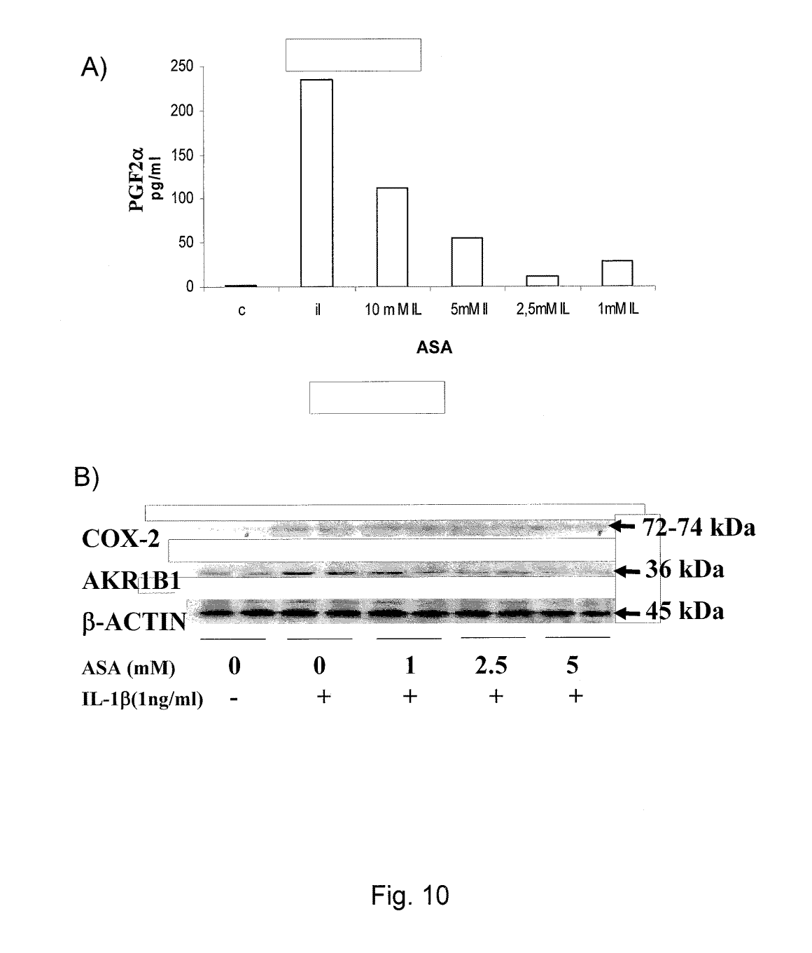

The PGFS activity of AKR1B1 therefore predominates over the reduction of glucose or peroxylipids (FIG. 3). However, the inhibitory effect of high glucose levels on PGFS activity (FIG. 9) confirms that it is a competitive substrate for AKR1B1, and that it may in fact constitute one of the pathogenic mechanism. In addition, when cells expressing AKR1B1 are treated with ASA, the protein expression level of AKR1B1 and its PGF synthase activity are strongly reduced (FIG. 10). This direct action of ASA on AKR1B1 suggests an additional site of action explaining the unique efficiency of ASA for protection against cardiac ischemia.

Similarly, the recently developed COX inhibitors Celebrex™ and Vioxx™ are COX-2 selective inhibitors that have proven extremely efficient to reduce pain and inflammation induced by PGE₂. Unfortunately, the use of several COX inhibitors has been found to be associated with an increased risk of heart failure, whereas other common NSAIDs acting indistinctly on both COXs, such as naproxen, do not induce such cardiovascular side-effects, although they often induce gastrointestinal side-effects.

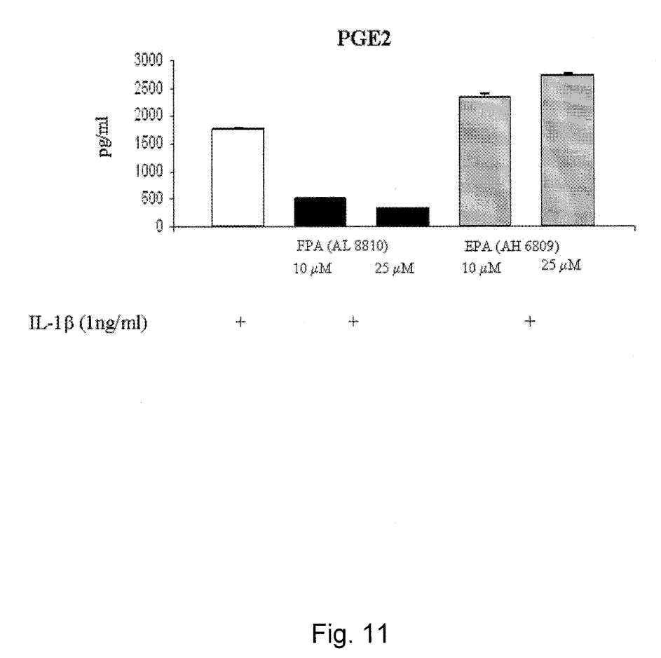

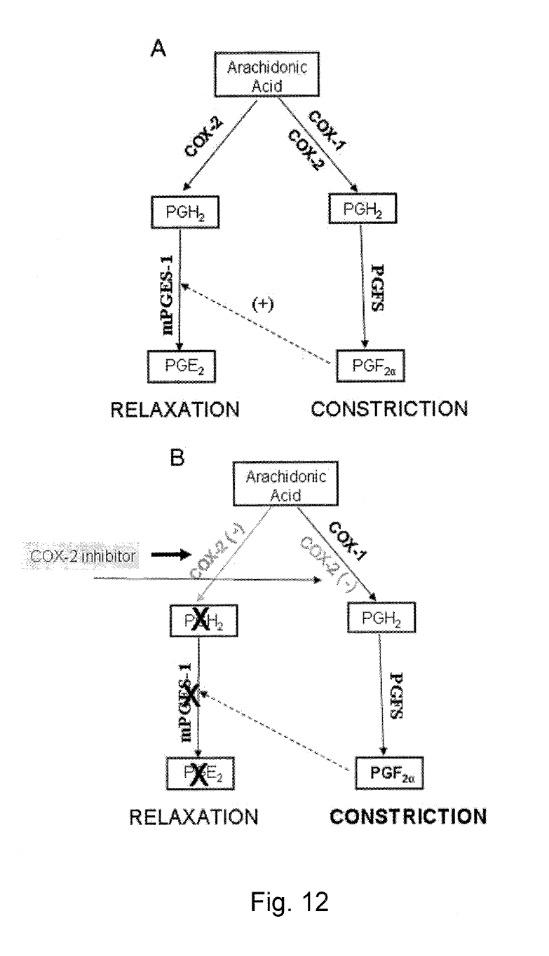

In the present study, we have clearly established an association between AKR1B1 expression, PGF_{2α} production and the stimulation of PGE₂ production in human endometrial stromal cells stimulated by IL-1β (FIG. 7 and FIG. 11). Previously, a cDNA microarray study of 15164 sequence-verified clones has identified AKR1B1 as an important gene upregulated by IL-1β in human endometrial cells (Rossi M et al., Reproduction, 2005, 130: pp 721), confirming our observation that it is a key inducible endometrial protein. The induction of PGE₂ is therefore a feedback mechanism compensating for PGF_{2α} overproduction that is mediated through the FP receptor of PGF₂₀ (FIGS. 7 and 12).

COX-2-specific inhibitors such as Vioxx™ are very efficient anti-inflammatory and anti-pain molecules. These drugs act preferentially by blocking the COX-2 pathway, which lowers the PGH₂ available as a substrate for mPGES-1, thus decreasing the production of PGE₂. In subjects having aberrantly high AKR1B1 levels, for example in subjects affected with a metabolic disorder such as type 2 diabetes, insulin resistance or obesity, and consequently high PGF_{2α} levels, a compensatory mechanism induces PGE₂ production in order to maintain an equilibrated PGF_{2α}/PGE₂ balance. However, if the same subjects have chronic pain in addition to their metabolic disorder, and these subjects are prescribed a COX-2-specific inhibitor for their chronic pain, PGF_{2α} levels will rise to uncompensated pathogenic levels because the feedback mechanism involving mPGES-1 and subsequent production of PGE₂ is rendered inoperative from the action of the COX-2-specific inhibitor. The resulting unbalanced PGF_{2α}/PGE₂ ratio can in turn induce a risk of cardiovascular ischemia caused by higher levels of PGF_{2α} in the heart.

Since the feedback mechanisms to regulate the balance between PGF_{2α} and PGE₂ are impaired, it is thus imperative that a reduction in PGF_{2α} must be performed in such a subject to prevent the cardiovascular side-effects. Such a regulation can be performed via PGF_{2α} inhibitors, such as, but not limited to, inhibitors of PGF_{2α} synthesis and inhibitors of PGF_{2α} binding to receptors (FP, EP1 and EP3 receptors). Readily available inhibitors of AKR1B1 could be evaluated for their potential utility, despite the fact that these inhibitors have originally been designed for blocking the polyol activity of AKR1B1, and not the PGFS activity. Since AKR1B1 activity is regulated at two different molecular locations, the polyol and PGFS activities may be affected according to totally distinct dynamics. Therefore, the use of an already available AKR1B1 (polyol) inhibitor for blocking the PGFS activity of AKR1B1 is not recommended prior to testing for their capacity to block the novel PGFS activity of AKR1B1, because there are no guarantee that this novel activity will be blocked.

As used herein, the expression “PGFS activity” is intended to encompass a prostaglandin F synthase enzymatic activity as traditionally involving the transformation of PGH₂ into PGF_{2α}. A molecule having a PGFS activity, such as ARK1B1, is therefore intended to reflect on the ability of this molecule to catalyze the enzymatic transformation of PGH₂ into PGF_{2α}. The result of a molecule having a PGFS activity, provided it is contacted with the adequate substrate in the adequate conditions to exert its activity, is the production of PGF variants, such as PGF_{2α}. This is reflected, in the case of PGF_{2α}, by an augmentation of the PGF_{2α} levels in the immediate environment of the molecule, or by an augmentation of its stable metabolites, such as PGFM, in blood circulation or urine.

The activity level of AKR1B1 is reflective of its PGFS activity in a biological environment, such as in a subject, having access to its PGH₂ substrate, and transforming this substrate into PGF_{2α}. The activity level of PGF_{2α} is reflective of its action in a biological environment, such as in a subject, exerting its action directly or via a receptor. The expression level of AKR1B1, or of PGF_{2α}, is reflective of the expression of the gene of AKR1B1, or of PGF_{2α}, that is reflected on the level of AKR1B1 mRNA or protein, or of PGF_{2α}, or of its stable metabolite PGFM. Measurements of expression levels and activity levels are performed according to techniques known in the art.

The expression “PGF/PGE ratio” as used herein is intended to encompass the ratio of prostaglandin F and its variants relative to prostaglandin E and its variants. While this ratio can be expressed as an activity ratio or an expression ratio, it is mainly intended to represent a concentration ratio. Examples of prostaglandin F variants include PGF_{1α}, PGF_{2α}, PGF_{3α}, and PGFM, while examples of prostaglandin E variants include PGE₁, PGE₂, PGE₃, and PGEM. It will be understood that if a ratio is to be used with the concentrations of specific PGF variants, such as both PGF_{2α} and PGFM for example, the ratio must be expressed in relation with concentrations the corresponding PGE variants, such as PGE₂ and PGEM in this example, for the ratio to be consistent within the present invention. Further, the PGF/PGE ratio can include almost exclusively PGFM and PGEM, with virtually no PGF_{2α} or PGE₂, such as in the case of a PGF/PGE ratio measured in a urine sample for example, wherein most if not all of the native prostaglandins (PGF_{2α} and PGE₂) have been degraded before reaching the bladder.

The terms “treatment”, “treating” and the like as used herein are intended to encompass any kind of action performed to a subject having the effect of reducing or removing a cause or a symptom of a condition as defined in the text, including, but not limited to, the administration of a molecule (such as a AKR1B1 inhibitor, a PGF_{2α} agonist, a PGF_{2α} receptor blocker, etc) to the subject.

The terms “administering”, “administration” and the likes as used herein are intended to encompass the administration of the substance of interest into a site of interest in the subject by any means known in the art and suitably adaptable to the substance to be administered. For example, a pharmaceutical composition containing the substance of interest, such as an AKR1B1 inhibitor or a PGF_{2α} receptor blocker for example, can be administered by parenteral, topical, oral, nasal, intrathecal, or local (e.g. as a cream or topical ointment) routes. Preferably, the administration is performed parentally, e.g., intravenously, subcutaneously, intradermally, or intramuscularly. In addition, it will be understood that for the administration of a pharmaceutical composition comprising the substance of interest, the substance of interest has to be dissolved or suspended in an acceptable carrier, preferably an aqueous carrier. To that effect, a variety of aqueous carriers may be used, such as for example water, buffered water, 0.8% saline, 0.3% glycine, hyaluronic acid and the like. These compositions may be sterilized by conventional, well-known sterilization techniques, or may be sterile filtered. The resulting aqueous solutions may be packaged for use as is, or lyophilized, the lyophilized preparation being combined with a sterile solution prior to administration. In addition, the compositions may contain pharmaceutically acceptable auxiliary substances as required to approximate physiological conditions, such as pH-adjusting and buffering agents, tonicity adjusting agents, wetting agents, preservatives, and the like, for example, sodium acetate, sodium lactate, sodium chloride, potassium chloride, calcium chloride, sorbitan monolaurate, triethanolamine oleate, etc.

The terms “prevention”, “preventing” and the like as used herein are intended to encompass any kind of action performed to a subject having the effect of preventing, stopping or slowing the progression of a condition as described in the text, including, but not limited to, the administration of a molecule (such as a AKR1B1 inhibitor, a PGF_{2α} antagonist, a PGF_{2α} receptor blocker, etc) to the subject. The prevention can be performed in a subject in which the condition has never developed, has started to develop, or is expected to develop.

The terms “prediction”, “predicting” and the like as used herein are intended to reflect on the determination of the risk of a subject to develop a condition, a disorder or a symptom. The prediction can be performed on a normal subject not affected by the condition, disorder or symptom, or on a subject affected by the condition, disorder or symptom, a prediction in the latter case being reflective on the evolution of the condition, disorder or symptom in response to the absence or presence of a treatment.

The terms “diagnosis”, “diagnosing” and the like as used herein are intended to reflect on the identification of a condition, a disorder or a symptom in a subject based on the determination of a physiological parameter, such as but not limited to the expression level of AKR1B1 or the activity level of AKR1B1, and the comparison of that same physiological parameter obtained from a subject known not to be affected by that condition, disorder or symptoms, or with a standard value for that particular physiological parameter.

The terms “alleviation”, “alleviating” and the like as used herein are intended to represent the removal, partial or total, of a side-effect normally occurring as a result of a COX treatment. The alleviation can be partial or total.

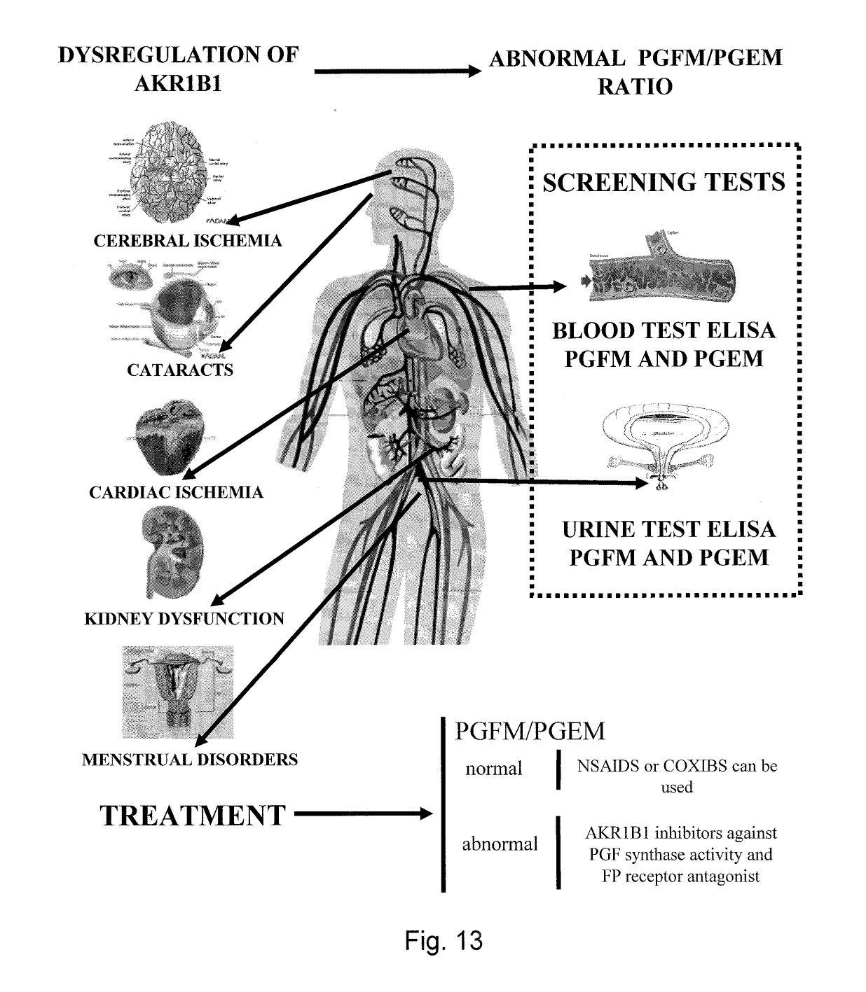

The conditions associated to an increase of PGF_{2α} levels as mentioned herein are intended to encompass any kind of condition, disorder or symptom that can be clearly correlated with a general or local increase in PGF variants levels, such as PGF_{2α} levels. Non-limitative examples of such conditions include metabolic disorders, obesity, type 2 diabetes, insulin resistance. Additional non-limitative examples of such conditions include cardiac ischemia, cerebral ischemia, bronchial constriction, kidney dysfunction. Further non-limitative examples of such conditions include menstrual pain or premature labor (FIG. 13). The conditions associated to a decrease of PGF_{2α} levels are intended to encompass any kind of condition, disorder or symptom that can be clearly correlated with a general or local decrease in PGF variants levels, such as PGF_{2α} levels. Non-limitative examples of such conditions include hyperglycemia, inflammation and impaired renal function.

The side-effects associated with the use of a COX inhibitor, or COX inhibitor-associated side-effects, as mentioned herein are intended to encompass any kind of condition, disorder or symptom associated with the use of a COX inhibitor that appeared as a direct or indirect consequence of the COX inhibitor use. These side-effects can be associated with either the dosage or the duration of the COX inhibitor treatment, while the severity of the side-effect is not necessarily directly associated to the dosage or the duration of the COX inhibitor treatment. Non-limitative examples of side-effects associated with the use of a COX inhibitor include cardiovascular side-effects, respiratory side-effects, cardiac ischemia, cardiac failure, cerebral ischemia, polyneuropathy, vision disorder, visual perception trouble, kidney dysfunction, menstrual disorders, heartburn, nausea, vomiting, stomach pain, swelling of foot, swelling of ankle, joint pain, muscle pain, weakness, bleeding, persisting sore throat, diarrhea, headache and fever.

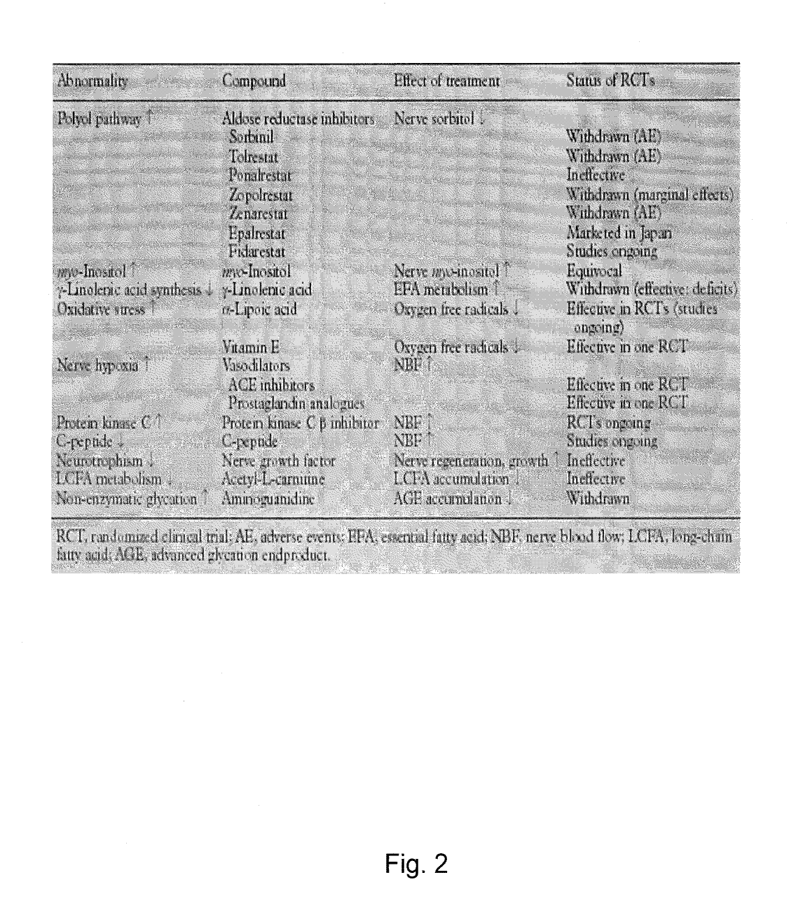

The expression “ARK1B1 inhibitor” as used herein is intended to encompass any molecule that can inhibit or lower AKR1B1 ability to exert its PGFS activity. Molecules exclusively inhibiting or lowering the transformation of glucose into sorbitol by AKR1B1, without affecting its ability to convert PGH₂ into PGF_{2α}, or any other PGH variants into its respective PGF variant, are not meant to be included within this term as used herein. Molecules inhibiting or lowering the transformation of glucose into sorbitol by AKR1B1, and also inhibiting or lowering, partially or totally, the ability of AKR1B1 to convert PGH₂ into PGF_{2α}, are meant to be included within this term as used herein. Known AKR1B1 inhibitors, such as Sorbinil™, Tolrestat™ and Zopolrestat™, or other AKR1B1 inhibitors mentioned in FIG. 2, for example, may be used, provided they inhibit or lower, partially or totally, the PGFS activity of AKR1B1. Inhibitors of AKR1B1 transcription, translation or post-translational modifications are encompassed within this expression since they prevent AKR1B1 from exerting its PGFS activity by blocking its synthesis. Regulators of AKR1B1 transit within the cytoplasm are included as long as they can lower the formation of PGF_{2α} from PGH₂ by AKR1B1. Activators of AKR1B1 degradation are also included since they lower the AKR1B1 levels available to form PGF_{2α}. It will be understood that the inhibition from these inhibitors can be total or partial, as well as can directly affect the ability of an AKR1B1 molecule to produce PGF_{2α} or generally inhibit, totally or partially, the production of PGF_{2α} by AKR1B1 in a biological system. Non-limitative examples of AKR1B1 inhibitors include AKR1B1-specific siRNA and AKR1B1-specific antibodies.

The expression “ARK1B1 activator” as used herein is intended to encompass any molecule that can increase or stimulate AKR1B1 ability to exert its PGFS activity. Molecules exclusively increasing or stimulating the transformation of glucose into sorbitol by AKR1B1, without affecting its ability to convert PGH₂ into PGF_{2α}, are not meant to be included within this term as used herein. Activators of AKR1B1 transcription, translation or post-translational modifications are encompassed within this expression since they increase AKR1B1 levels available to exert a PGFS activity by stimulating its synthesis. Regulators of AKR1B1 transit within the cytoplasm are included as long as they increase the formation of PGF_{2α} from PGH₂ by AKR1B1. Inhibitors of AKR1B1 degradation are also included since they prevent the lowering of the AKR1B1 levels available to form PGF_{2α}. It will be understood that the increase or stimulation of these inhibitors can directly affect the ability of an AKR1B1 molecule to produce PGF_{2α} or generally increase the whole production of PGF_{2α} by AKR1B1 in a biological system. Non-limitative examples of AKR1B1 activators include AKR1B1 gene, vector containing an AKR1B1 gene for at least the portion of the gene encoding for the PGFS activity, AKR1B1 protein and AKR1B1 peptide having the PGFS activity of the AKR1B1 protein.

The term “COX inhibitor” as used herein is intended to encompass any molecule inhibiting the expression of one or more COX enzyme gene, or the action of one or more COX enzyme protein. The COX gene and protein can be any COX, including COX-1, COX-2 or COX-3.

The expression “receptor blocker” as used herein is intended to encompass any molecule blocking the normal action or signaling pathway of a receptor prior or following the binding of its ligand, either by preventing the ligand to bind to the receptor, by preventing the receptor to bind to its ligand, by preventing the ligand-receptor complex from activating its second messenger system, or by preventing the ligand-receptor complex from being internalized in the cell. The receptors aimed to be blocked in the present application can be, for example, receptors having a sufficient affinity with PGF_{2α} for binding with PGF_{2α} and exerting the biological effect of PGF_{2α}. Non-limitative examples of such receptors include FP receptor, EP1 receptor and EP3 receptor. Non-limitative examples of receptor blockers include FP receptor blocker, EP1 receptor blocker and EP3 receptor blocker.

The term “PGF_{2α} antagonist” as used herein is intended to include any molecule that can bind to a PGF_{2α} receptor in place of PGF_{2α}, or that is capable of blocking a biological effect of PGF_{2α}. A PGF_{2α} antagonist can compete for a binding site with an endogenous PGF_{2α}, thus preventing the endogenous PCF_{2α} from exerting its effect.

The expression “subject” as used herein is intended to encompass any mammalian subject, such as for example a human or a dog.

The expression “biological fluid” as used herein is intended to encompass any fluid originating from a mammalian organism, such as for example blood, plasma, urine, saliva, sweat, and menses.

### Example 1

### The Human AKR1B1 Qualifies as a Functional PGFS in the Endometrium

In the bovine endometrium, we previously demonstrated a strong PGFS activity of AKR1B5 recently renamed as bos taurus AKR1B1 (Gene ID: 317748), a new function for this enzyme previously known for its 20a-HSD and glucose metabolism activities (Madore et al., J Biol Chem 278(13); 11205-12, 2003). The human and bovine AKR1B1 both belong to the aldoketoreductase 1B family and share 86% identity or homology. The human AKR1B1 (Gene ID: 231) also known as the aldose reductase is highly expressed in the placenta for glucose metabolism and in the eye and kidney for osmotic regulation.

After identifying the bovine AKR1B1 as a functional PGFS, we have found that AKR1B1 expression was associated with PGF_{2α} production in human endometrial cell lines and in decidualized stromal cells (Chapdelaine et al., Mol Hum Reprod, 12(5); 309-19, 2006). However, expression of AKR1B1 within the human endometrium and its ability to act as a PGF synthases to produce PGF_{2α} remain to be investigated. Therefore, in the present study, we have studied the expression of both AKR1B1 and AKR1C3 at the mRNA and protein levels in non pregnant human endometrium across the menstrual cycle. We have also investigated their ability to produce PGF_{2α} using human endometrial cell lines.

Endometrial biopsies were taken from women aged between 25 to 50 years with regular cycles (21-35 days) without hormonal treatment in the 3 months prior to biopsy collection and undergoing gynecological investigation for infertility or menorrhagia. Informed consent for donation of anonymous endometrial samples was obtained before tissue collection. Biopsies representing functionalis layer were collected with an endometrial curette (Pipelle) and dated according to the stated last menstrual period. The stage of the cycle (proliferative or secretory) was then confirmed by histological examination using the criterion of Noyes (Noyes et al., Fertil Steril 1; 3-25, 1950) and samples with conflicting dating were discarded. Shortly after collection, the tissue was put in cold Hank's solution, placed on ice and brought to the laboratory. The samples were washed, divided and portions processed differentially for RNA and protein analysis.

Analysis of AKR1B1 and AKR1C3 mRNAs was performed by competitive PCR. Briefly, biopsies (N=48) were processed immediately upon reception. RNAs was prepared in TRIzol™ Reagent according to the manufacturer's instructions and samples stored at −80° C. until used for competitive PCR analysis. To generate RNA template competitors, a 100 bp deletion was created in AKR1B1 cDNA contained in pEF6/V5 by cutting with Hpa1 (containing 2 restrictions sites) and self ligation while for AKR1C3 cDNA, a 150 bp deletion was done with Ppum1 and Bsg1 blunted with Klenow followed by self ligation. The resulting recombinants were linearized with Pme1, transcribed into RNA with T7 RNA polymerase, purified on a polyacrylamide gel and RNA quantified at 260 nm.

For competitive PCR analysis of endometrial RNA (20 μg), cDNA first strands were synthesized in presence of AKR1B1 or AKR1C3 RNA competitors with Superscript II™ reverse transcriptase using the following primers: AKR1B1 (344 by amplicon): forward 5′-gatgagtcgggcaatgtggttcc-3′ (SEQ ID NO:1) and reverse 5′-cttggctgcgatcgccttgatcc-3′ (SEQ ID NO:2); AKR1C3 (565 by amplicon): forward 5′-ctaaagccaggtgaggaactttc-3′ (SEQ ID NO:3) and reverse 5′-ctatcactgttaaaatagtggag-3′ (SEQ ID NO:4). PCR amplification was achieved as follows: 94° C. for 20 seconds, 55° C. for 30 seconds and 72° C. for 30 seconds during 35 cycles. Five RTs with different competitor concentrations were performed for both enzymes and PCR products were loaded on 1.5-1.7% agarose gel stained with ethidium bromide and bands quantified by image analysis using the AlphaImager 2000™ software (Alpha Innotech Corporation, San Leandro, Calif.).

For immunohistochemistry, 3 μm tissue sections of human endometrium were taken at different periods of the menstrual cycle, fixed in 4% paraformaldehyde and prepared as paraffin-embedded sections. Slides were deparaffinized in xylene and rehydrated using decreasing grades of ethanol. Endogenous peroxidase activity was blocked with 3% H₂0₂ in methanol. Antigen retrieval was done by heating the sections in 1M urea solution for 15 minutes in a microwave oven at medium power. Tissue sections were then blocked with 10% goat serum for 1 hour in a humidified chamber at room temperature followed by an overnight incubation at 4° C. with primary antibodies at optimal dilutions (AKR1B1 1:250 in-house rabbit anti-human), AKR1C3 1:200 (goat polyclonal, Abcam inc., Cambridge, Mass., USA), COX-1 1:500 (rabbit, kindly provided by Dr. S. Kargman, Merck, QC, Canada) and COX-2 1:750(rabbit, kindly provided by Dr. S. Kargman, Merck, QC, Canada)). Non-immune rabbit serum was used as the negative control. The next day, sections were washed in PBS and incubated 30 minutes at room temperature with biotinylated goat anti-rabbit IgG 1:200 (AKR1B1, COX-1 and -2) or rabbit anti-goat IgG 1:200 (AKR1C3) as secondary antibodies (Dako Diagnostic of Canada inc., Mississauga, ON, Canada). After washing with PBS, sections were treated with avidin-biotin-peroxidase complex (Vectastain™ Elite ABC kit, Vector Laboratories Inc., Burlingame, Calif., USA) followed by staining with 3-amino-9-ethyl carbazole. Finally, sections were washed with water and counterstained with Harris hematoxylin reagent (Sigma, Mississauga, Canada). The staining was evaluated subjectively by three blinded observers not involved with the present study, using a scoring system of immunostaining intensity interpreted as absent (O), weak (I), moderate (2), or intense (3). Individual scores for each slide were averaged and expressed as relative expression level.

Specific short interfering RNAs (siRNAs) for AKR1B1 were designed using the TROD (T7 RNAi Oligo Designer) software v. 1.1.2 (Dudek and Picard, Nucleic Acids Res, 32(Web Server issue); W121-3, 2004) designed for facilitating the identification of optimal oligonucleotides for the production of siRNA, with T7 RNA polymerase forward: 5′-aaattgttgagcaggagacggctatagtgagtcgtattacc-3′ (SEQ ID NO:5) and reverse: 5′-aagccgtctcctgctcaacaactatagtgagtcgtattacc-3′ (SEQ ID NO:6), according to the procedure of Donze (Donze et al., nucleic acid research, 2002, 30: e46) with RiboMax™ polymerase kit (Promega, Madison, Wis., USA). The resulting siRNA products were purified by ethanol precipitation and 100 ng/ml were used for transfection of cells grown in 6 or 24-well plates using Lipofectamine™ 2000 (Invitrogen).

Western blot analysis was performed with approximately 20 μg total proteins from cultured cells on a 10% SDS-PAGE gel, followed by electro-transfer onto nitrocellulose membrane. The primary antibodies used for the present study were rabbit AKR1B1 (dilution 1:1000) and COX-2 (dilution 1:10 000) anti-sera and goat AKR1C3 (dilution 1:500) anti-serum. A 8-actin monoclonal antibody (1:5000, Sigma, Mississauga, Ontario, Canada) was used as an internal control. Goat anti-rabbit IgG conjugated with horseradish peroxidase (HRP) (Jackson Immunoresearch Laboratories, West Grove, Pa., USA), rabbit anti-goat IgG HRP and goat anti-mouse IgG HRP were used as secondary antibodies. Chemiluminescence was analyzed with autoradiography films at optimal times of exposure following treatment of the membranes with Renaissance™ reagent (NEN Life Science Products, Boston, Mass., USA).

Northern blot analysis was performed with 20 μg total RNA from endometrial cells in culture on a 1.2% formaldehyde-agarose gel. Following electrophoresis, RNA were transferred overnight onto a nylon membrane in 10× saline-sodium citrate (SSC). The AKR1B1 cDNA probe was generated by labeling the −500 by cDNA fragment with [α-³²P]dCTP (3000 Ci/mmol) (Perkin-Elmer Life Sciences, Markham, ON, Canada) using the Ready-To-Go™ DNA labeling Kit (Amersham/Pharmacia). Prehybridization (2-4 hours) and hybridization (overnight) were performed at 45° C. using UltraHyb™ solution (Ambion Inc., Austin, Tex., USA). Blots were then washed twice at 65° C. for 15 minutes in 0.5×SSC and exposed on BioMAX™ films for quantification. 18S ribosomal RNA was used to confirm uniform loading of RNA samples.

For cell culture transfection, immortalized human endometrial stromal cells (HIESC-2) and epithelial cells (HIEEC-22) were cultured in RPMI 1640 without phenol red, containing 50 IU penicillin-streptomycin supplemented with 10% whole fetal bovine serum (FBS). Ten percent dextran-coated charcoal-extracted FBS was used once cells have reached confluency. Knock-down and knock-in transfections of cells with AKR1B1 specific siRNA, AKR1B1 or AKRIC3 cDNAs in pCR3.1 expression vectors were performed with Lipofectamine™ 2000 for 4 hours in culture medium without antibiotic. Thirty-six hours after transfection (24 hours for siRNA transfection), cells were treated for 24 hours with recombinant human interleukin 1β (IL-1β) (1 ng/ml) (R&D Systems, Minneapolis, Minn., USA) or arachidonic acid (AA) 10 μM in RPMI 1640 medium without serum. At the end of the treatment period, the culture medium was recovered and stored at −20° C. until evaluation for PGF_{2α} production.

For evaluation of AKR1B1 enzymatic activity by thin layer chromatography (TLC), recombinant AKR1B1 protein was overexpressed in Escherichia coli, purified, and the enzymatic activity was determined by inserting AKR1B1 in the NdeI restriction site of pET17B. HIS-TAG proteins were produced and purified on Nickel-sepharose column (Novagen). Enzymatic activity was measured by monitoring NADPH degradation at 340 nm. The assays were performed in 1 ml of 50 mM Tris-HCl pH 7.5, 100 μM NADPH with 10 to 100 μg of enzyme and various concentrations of PGH₂. Migration was performed in ethyl acetate [110:50:20] water saturated solvent and detection of PGF_{2α} production was achieved by spraying the TLC silica plates with phosphomolybdic acid 10% (v/v) in methanol and cooking the plate at 120° C. for 10 minutes.

Enzymatic immunoassay (EIA) was performed with an acetylcholinesterase-linked PGF_{2α} tracer (Cayman) as described previously (Asselin et al., Biol Reprod 54(2); 371-9, 1996). Sheep anti-PGF_{2α} (Bio-Quant, Ann Arbor, Mich., USA) was used as the selective antibody. Inter-assay and intra-assay coefficients of variations (n=12) were of 16% and 10% respectively. Statistical analysis was performed using ANOVA with Statview™ software (Abacus Concept, California). Values were considered statistically significant for p<0.05.

Analysis of mRNA expression for COX-1, COX-2, AKR1B1 and AKR1C3 was performed by competitive RT-PCR in endometrial biopsies collected at different period of the menstrual cycle (FIG. 4). The results show that COX-1 mRNA expression was higher during the secretory phase (FIG. 4A), that of COX-2 lower than COX-1 and higher during the proliferative phase (FIG. 4B), AKR1B1 mRNA expression was highest during the late secretory phase and menses (FIG. 4C) and by comparison, the relative expression of AKR1C3 mRNA was lower and did not vary across the menstrual cycle (FIG. 4D).

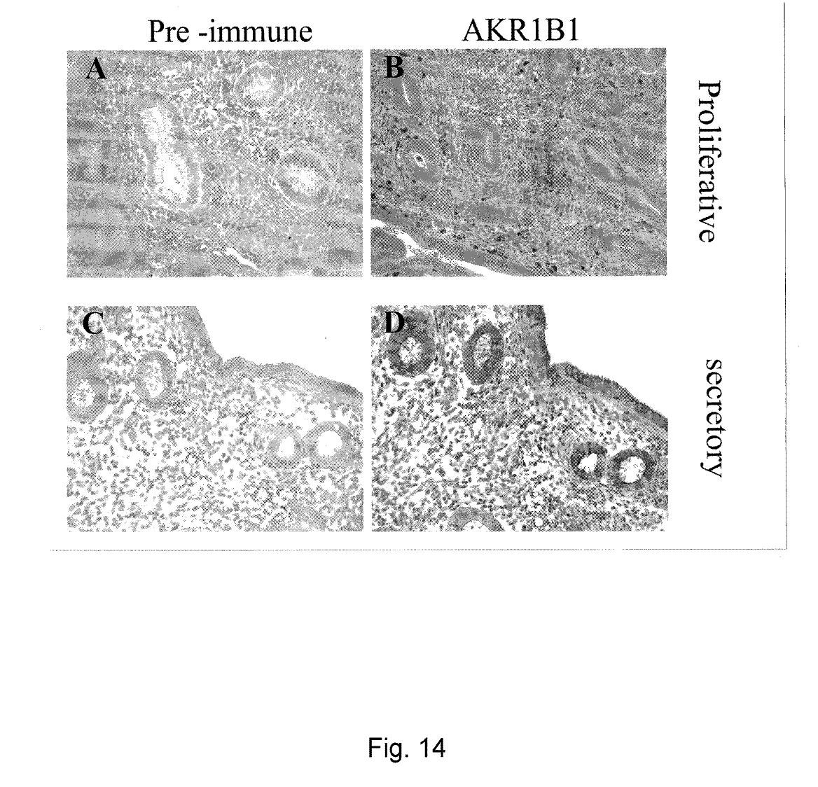

Immunohistochemical staining for AKR1B1 and AKR1C3 was performed in endometrial samples collected at different phases of the menstrual cycle (FIG. 14). AKR1B1 protein is present in luminal and glandular epithelial and in stromal cells of the endometrium. When staining was evaluated by subjective analysis, higher expression was found in early proliferative and mid secretory compared with others phases of menstrual cycle in both epithelial and stromal cell compartments (not shown). The pattern of AKR1B1 protein expression correlates with that of mRNA expression during the menstrual cycle. AKR1C3 protein expression exhibits constant staining across the menstrual cycle (not shown) as was observed for mRNA expression (FIG. 4D). By contrast with AKR1B1, AKR1C3 staining is completely absent in stromal cells and localized mainly in luminal and glandular epithelial cells. Immunohistochemical localization of COX-1 and COX-2 was performed on the same samples used for AKR1B1 and AKR1C3.

The effect of IL-1β, a known regulator of PG production, on AKR1B1 and AKR1C3 protein and their relative contribution to produce PGF_{2α} was studied in cultured endometrial cells. Western blot analysis shows that when the endometrial cell lines HIESC-2 and HIEEC-22 are treated with IL-1β (1 ng/ml) an increase of COX-2 protein level is associated with an increase of AKR1B1 protein (FIG. 5) The use of AKR1B1 specific siRNA in HIESC-2 induces a significant decrease of AKR1B1 mRNA and protein without reduction of mPGES-1 (FIG. 8C) or COX-2 (FIG. 6A) protein following treatment with IL-1β. Under the same conditions, β-actin does not vary. The decrease in AKR1B1 protein by specific siRNA knock-down was associated with a significant reduction of PGF_{2α} production (P<0.05) (FIG. 6B). In accordance with immunohistochemical localization, we were unable to detect AKR1C3 protein in the stromal cell line (HIESC-2) by Western analysis, but transfection of AKR1C3 in these cells induces a detectable immunoreaction (FIG. 6C). AKR1C3 protein was easily detectable in HIEEC-22 by Western blot analysis but treatment with IL-1β (1 ng/ml) has no effect on its expression level (FIG. 6C).

For analysis of the PGFS activity of AKR1B1, AKR1B1 recombinant protein was produced in Escherichia coli and purified on a nickel-nitrilotriacetic column. The recombinant protein was found to functionally reduce phenanthrenequinone and NADPH at a rate of 10 nmole/min/mg in presence of 40 μM PGH₂ as monitored by absorbance at 340 nm (FIG. 8A). The conversion of PGH₂ into PGF_{2α} was confirmed by TLC in which a spot corresponding to the PGF_{2α} marker is detected (FIG. 8A). To confirm PGF synthase activity, AKR1B1 full length cDNA expressed under the CMV promoter was transfected in HIESC-2 cells. Treatment of the transfected cells with 10 μM AA results in increased production of PGF_{2α} in the culture medium (FIG. 8B), by contrast with what was observed when AKR1B1 is knocked down using specific siRNA (FIG. 6A). Together, these observations confirm AKR1B1 as a functional PGF synthase

Prostaglandins are important regulators of female reproductive function and contribute to gynecological disorders. Normal menstruation depend on an equilibrium between vasoconstrictors such as PGF_{2α} and vasodilators such as PGE₂ or nitric oxide (NO). Excessive production of contracting prostaglandins create an ischemia-reperfusion response causing painful menstruation or dysmenorrhea, whereas increased vasodilatation leads to abundant menstrual bleeding. NSAIDs represent the most important and widely used drugs on the market and they are all efficient to treat menstrual disorders at some level. However these drugs act at an early step of biosynthesis common for all PGs and not only the isotype responsible for the pathological response. Because of its notorious role on inflammation and pain, the biosynthetic pathway leading to PGE₂ has been studied extensively, but that of PGF_{2α} is poorly documented. The data presented describes for the first time the expression of two gene candidates, AKR1B1 and AKR1C3, and the corresponding proteins, and their functional association with PGF_{2α} production.

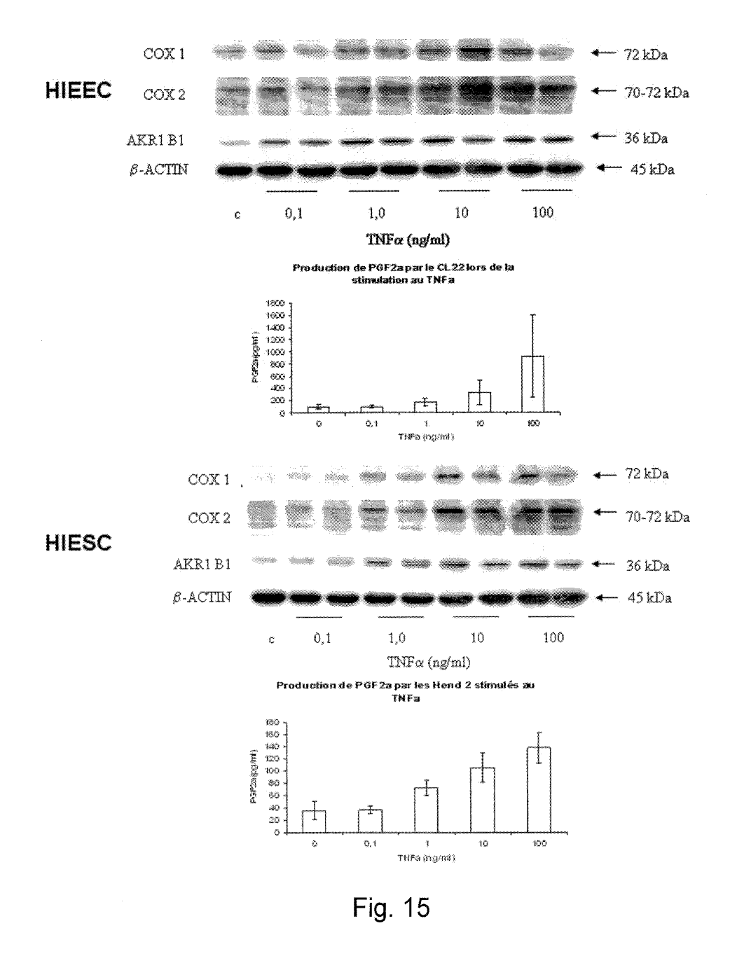

In the human endometrium, it has been reported that production of PGF_{2α} is higher in late secretory and menstrual periods of the menstrual cycle (Downie et al., J Physiol 236(2); 465-72, 1974). Accordingly, both AKR1B1 and AKR1C3 enzymes are present in the endometrium throughout the menstrual cycle. By contrast with AKR1B1 expressed in both stromal and glandular epithelial cells and modulated in accordance with endometrial PGF_{2α} production, AKR1C3 expression is constant and completely absent in stromal cells. The absence of the only currently accepted human PGFS, i.e. AKR1C3, in stromal cells was surprising because we and others have shown that human endometrial stromal cells produce high levels of PGF_{2α} that is further stimulated by cytokines such as IL-1β (FIG. 8, 6) and TNF-α (FIG. 15). Having shown that AKR1B1 was expressed in human endometrial cells and modulated in parallel with PGF_{2α} production, we investigated the potential PGFS activity of AKR1B1.

We have first demonstrated the ability of the purified recombinant human AKR1B1 to release PGF_{2α} and metabolize PGH₂ in vitro in presence of NADPH (FIG. 8A). The human AKR1B1 is thus able to metabolize PGH₂ and form PGF_{2α} with a high efficiency. In fact, AKR1B1 uses PGH₂ at concentrations well within the physiological range whereas it processes glucose only at supra-physiological concentrations found primarily under pathological conditions. It was then important to show that alterations in the expression of the AKR1B1 protein impacts on PGF_{2α} production. We have found that transfection of either epithelial or stromal cells with AKR1B1 induced increased production of PGF_{2α} (FIG. 8) whereas knocking down its expression with specific siRNA reduced PGF_{2α} production (FIG. 6). We have also confirmed the PGFS activity of AKR1C3 following transfection of endometrial stromal cells (FIG. 6C) where PGF_{2α} production is increased compared with non-transfected cells in presence of exogenous AA (results not shown). Because AKR1C3 is expressed only in epithelial cells (representing only a small fraction of endometrial functionalis) and since this enzyme is not modulated during the cycle nor stimulated by IL-1 (3, its contribution to the release of endometrial PGF_{2α} is probably negligible. IL-1β is an important regulator of endometrial PG production that also induces apoptosis in the epithelial cells of the endometrium, to initiate menstruation. Interestingly, a cDNA microarray study of 15164 sequence-verified clones has identified AKR1B1 as an important gene upregulated by IL-1β in human endometrial cells, supporting our observation that it is a key inducible endometrial protein (Rossi, et al., Reproduction 130(5); 721-9, 2005).

Together, these results show that AKR1B1 is the primary candidate to be considered as the functional PGFS responsible for PGF_{2α} production in the human endometrium (FIG. 3).

AKR1B1 has been previously studied, but its contribution to prostaglandin production had never been suspected. AKR1B1 has been traditionally associated with reduction of glucose and diabetes-induced oxidative stress. Accordingly, AKR1B1 knockout mice have been used to study the pathogenesis of various diseases associated with diabetes mellitus such as cataract, retinopathy, neuropathy and nephropathy (Ho et al., Mol Cell Biol 20(16); 5840-6, 2000). Interestingly, transgenic mice overexpressing human AKR1B1 were more prone to myocardial ischemic injury (Hwang et al. Faseb J 18(11); 1192-9, 2004), whereas knockout mice appeared protected against cerebral ischemic injury (Lo et al. J Cereb Blood Flow Metab 27(8); 1496-509, 2007). In hindsight, these observations are compatible with the involvement of AKR1B1 in the regulation of vascular tone by mechanisms distinct from glucose metabolism. Interestingly, this is a documented function of PGF_{2α} and its FP receptor (Norel, Scientific World Journal 7; 1359-74, 2007). If PGFS activity or FP receptors are altered in presence of high glucose levels or aberrant insulin response, it could explain the development of vascular and neurological complications in diabetes.

Because of their association with inflammation and other pathological conditions, prostaglandins as a whole are considered as foes. Moreover, because NSAIDs, a single class of medication, are highly efficient to treat pain, inflammation and menstrual disorders, PGs are treated globally as if they were a single factor. There are two limiting steps in the synthesis of PGs; the liberation of AA from membrane phospholipids by phospholipases and the generation of the intermediate PG metabolite PGH₂ by PGH synthases or COXs. These steps are common for all bioactive PGs and not limited a priori to the specific one that drives aberrant responses. PGs induce a wide variety of responses mediated by receptors distinct for each isoform and using several second messenger systems. In the vascular system, TXA₂ and PGI₂ exert opposing action on coagulation and vascular tone to regulate hemostasis. In the reproductive system the same is often observed for PGF_{2α} and PGE₂.

There have been reports showing that some terminal synthases are preferentially associated with a specific COX such as mPGES-1 with COX-2 or mPGES-2 with COX-1 (Ueno et al., Biochem Biophys Res Commun 338(1); 70-6, 2005). Intriguingly, in spite of significant and stimulus sensitive production of PGF_{2α}, no co-localization or association was found between COXs and PGF synthases (Nakashima et al., Biochem Biophys Acta 1633(2); 96-105, 2003). Such associations would imply that inhibition of a specific COX could exert some selectivity on the release of a specific PG. In this respect ASA, the first marketed NSAID (ASPIRIN™) exhibits a slight preference for COX-1 and platelets thus yielding preferential inhibition of TXA₂ over PGI₂ in the vascular system. Similarly, the recently developed COX inhibitors such as Bextra™ and Vioxx™ are COX-2 selective and have proven extremely efficient to reduce pain and inflammation induced by PGE₂ (Zeilhofer, Trends Pharmacol Sci 27(9); 467-74, 2006). Unfortunately, the use of these drugs has been found to be associated with an increased risk of heart failure whereas other common NSAIDs such as ibuprofen and naproxen, act on both COX with no distinction between COX-1 and COX-2 (Rainsford, Inflammopharmacology 13(4); 331-41, 2005). Therefore, acting at the level of terminal synthases responsible for the release of specific PG isotypes appears as a promising avenue to control the release of “bad” PGs while allowing the action of the “good” PGs.

AKR1B1 was first identified as a key enzyme of the polyol pathway and more recently as a detoxification enzyme involved in the reduction of a wide range of carbonyl compound including benzaldehyde derivatives, quinones, sugars and many lipid peroxidation end products such as 4-hydroxy trans-2-nonenal (HNE) and acrolein (Srivastava et al., Endocr Rev 26(3); 380-92, 2005). The present finding that AKR1B1 is a functional PGFS liberating PGF_{2α}, a bioactive metabolite acting on a specific receptor, was unexpected and is highly challenging.

### Example 2

### Retrovirus Infection and Establishment of SV40 TAG Cell Lines Expressing PGFS Activity

The retroviral vector SSR69 containing SV40 large TAG and a gene resistant to hygromycin was transfected with Effectene™ (Qiagen, Mississauga, ON, Canada) in the mouse amphotropic packaging cell line PA 317. The resulting colonies resistant to hygromycin (800 μg/ml, Roche, Mississauga, ON, Canada) were cultured, and the supernatants containing amphotropic viruses were collected and used to infect, separately, purified stromal and epithelial cells in primary culture. Endometrial cells grown in six-well plates were infected in the presence of polybrene (8 μg/ml, Sigma) for 6 h, and the procedure was repeated 24 h later. The day following the last infection, the cells were trypsinized and seeded in 10 mm dishes in the presence of hygromycin (400 μg/ml). The cultures were grown for 7-8 days until the TAG-infected cells formed colonies while control non-infected cells died in the presence of the antibiotic.

A total of 17 clones (17 colonies) for stromal cells and 50 for epithelial cells were picked by clonal selection (cloning o-ring) and grown in 24-well plates until confluency and then seeded in T-25 flasks. The PD for TAG clones was calculated as follows: n(PD)=log(final cells count)−log(inoculation cell count)/0.301. Because colonies are produced from a single cell, we calculated that at confluency, the initial PD in T-25 flask was 19.2. The TAG clones were maintained in complete culture medium unless specified differently. The clones were then selected according to their growth rate, production of PGs and response to IL-1β.

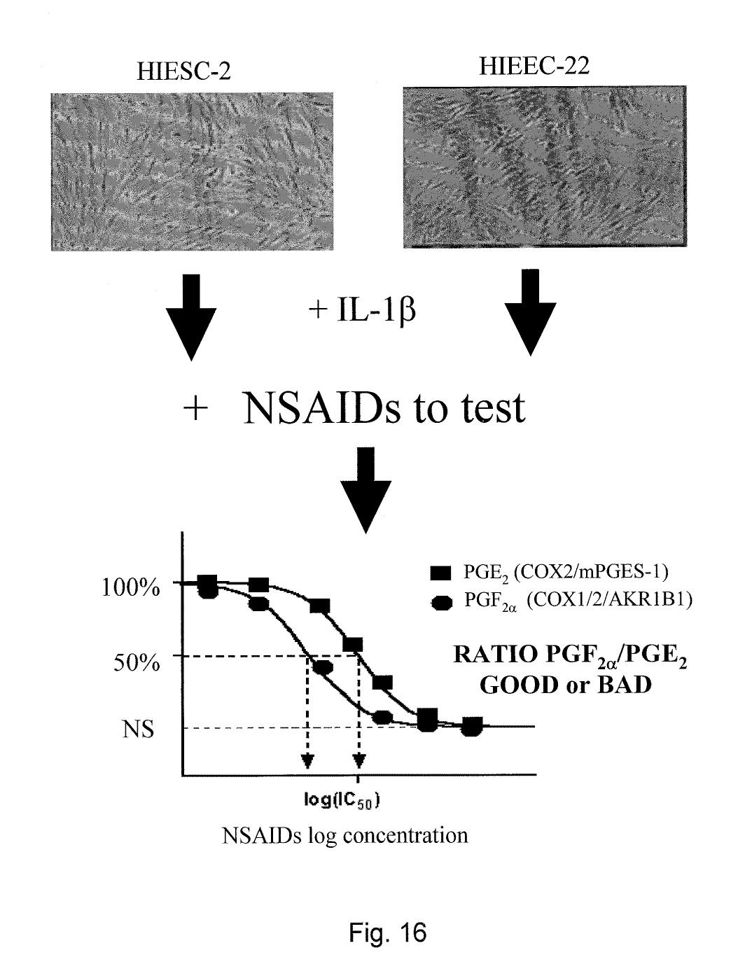

Two cell lines, one of stromal origin (HIESC-2) (IDAC deposit account number 301008-04) and one of epithelial origin (HIEEC-22) (IDAC deposit account number 301008-05), were selected and characterized thoroughly. Both cell lines produce significant levels of PGE₂ and PGF_{2α} that can be stimulated by IL1β. The epithelial cell line HIEEC-22 expresses the two PGFS AKR1C3 and AKR1B1 whereas HIESC-2 expresses only AKR1B1. In both cases, increased PGF_{2α} production is associated with increased expression of COX-2 and AKR1B1. Both cell lines are ideal models for testing the effect of COX inhibitors and NSAIDs on different PG isoforms in an integrated manner (FIG. 16), and that the relative effect of those drugs on the PGF/PGE ratio is predictive of their relative cardiovascular safety, and/or of their cardiovascular risk.

### Example 3

### Establishment of a Link Between AKR1B1 and COX-2-Inhibitor-Associated Increased Risk of Heart Failure

Following the discovery of AKR1B1 as a major PGFS involved in the synthesis of PGF_{2α}, and since AKR1B1 has been involved in diabetes-associated pathologies, and that its impact on cardiac and cerebral ischemia has been demonstrated in transgenic mice (Hwang Y. C. et al., 2004; Iwata K. et al., 2006; Vikramadithyan R K), we investigated if the PGFS function of AKR1B1 could allow for the identification of PGF_{2α} as a molecule responsible for ischemia and pain.

The PGFS activity of AKR1B1 therefore represents a crucial step in the synthesis of PGF_{2α} from PGH₂ released by COX-1 and COX-2. The COX-inhibiting NSAIDs are commonly used in the treatment of headaches and muscle aches. Prior art studies of AKR1B1 mainly focused on its role in polyols synthesis or in lipid detoxification. While AKR1B1 inhibitors have been developed to treat pathological conditions such as diabetic complications, ischemic damage of non-cardiac tissues, and Huntington's disease (U.S. Pat. No. 6,696,407, U.S. Pat. No. 6,127,367, U.S. Pat. No. 6,380,200), the identification of AKR1B1 as a PGFS has never been considered or used as an end issue. Our observation led us to consider AKR1B1 as a new alternate target to regulate PGF_{2α} output associated with pathologic conditions more selectively than NSAIDs used as COX-1 and COX-2-specific inhibitors.

NSAIDs inhibiting COX-1 induce ulcers and other gastric problems, while COX-2-specific inhibitors possess analgesic properties used in the treatment of pain associated with arthritis, rheumatism and inflammation. However, pharmaceutical companies have developed powerful analgesic agents specifically targeting COX-2 for treating arthritis and related disorders without presenting the gastric side effects induced by COX-1 inhibitors. Most of those products, such as Vioxx™ and Bextra™, have been withdrawn from the market for having an increased risk in causing infarctus as a side effect. However, Aspirin™ and ibuprofen (both NSAIDs) are still used for their good analgesic activity.

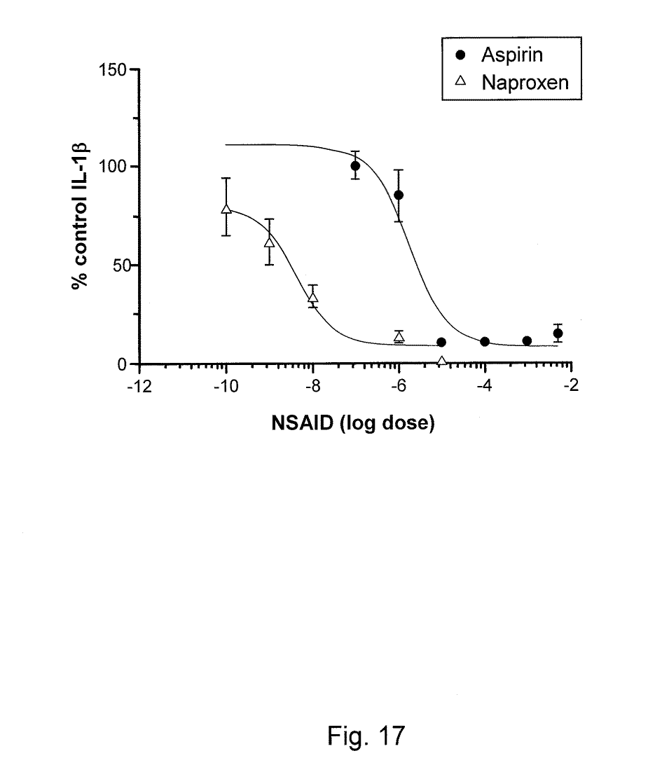

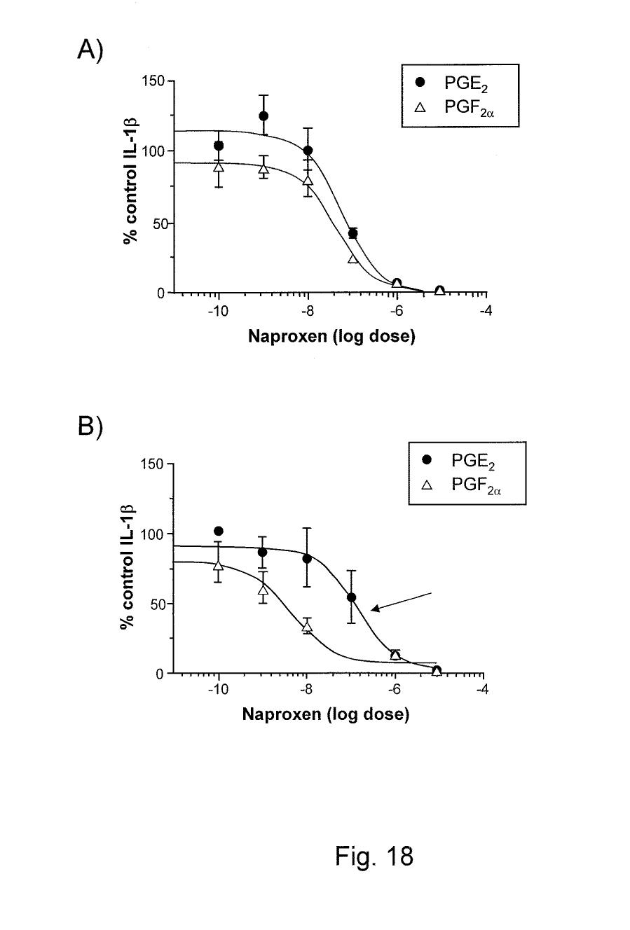

Considering that ASA (Aspirin™) is the only NSAID clinically proven to exert cardio-protective effects, we hypothesized that this drug could be interacting directly with AKR1B1. The results of a dose-dependent inhibition of AKR1B1 protein with Aspirin™ (1-5 mM) in our cellular model confirmed this hypothesis (FIG. 10). Naproxen, a NSAID known to inhibit both COX-1 and COX-2 enzymes, was shown to be more potent than Aspirin™ for the inhibition of PGF_{2α} production by endometrial epithelial HIEEC cells stimulated by IL-1β (FIG. 17). Moreover, naproxen was shown to inhibit PGF_{2α} and PGE₂ production by endometrial stromal cells (which only express COX-2) with comparable IC₅₀ (FIG. 18A), whereas it inhibits PGF_{2α} 100 times more efficiently than PGE₂ in endometrial epithelial cells (which express both COX-1 and COX-2) (FIG. 18B). At 10 nM naproxen thus induces a strong alteration of the PGF_{2α}/PGE₂ ratio in favor of PGE₂ (FIG. 18).

We also demonstrated that the PGFS activity of AKR1B1 was modulated by 25 mM of D-glucose, showing putative competition with PGH₂ at the catalytic site of the enzyme (FIG. 9). Further, overexpression of AKR1B1 in transgenic mice under normal glycemia has been associated with ischemic responses characteristic of a vasoconstrictor such as PGF_{2α}.

Because of the constitutive expression of COX-1 in most tissues and the induced release of AA and/or stimulation of COX-2 expression under many pathologic conditions, we herein propose that an increased expression of AKR1B1 induces an aberrant overproduction of PGF_{2α}. In response, but depending on tissues, PGF_{2α} overproduction can be compensated up to a certain point by the release of compensatory PGE₂ through a FP receptor-dependent mechanism (FIG. 7). We also propose that insulin resistance with normal glycemia triggers the overexpression of AKR1B1, and that administration of a COX-2-specific inhibitor under these conditions can favor aberrantly high PGF_{2α}/PGE₂ ratio that could in turn trigger ischemia in cardiac and other tissues. On the other hand, high glucose levels, which are typical of diabetes, reduce the PGF_{2α} production by AKR1B1 while inducing the release of sorbitol, thus increasing ocular pressure and altering renal function.

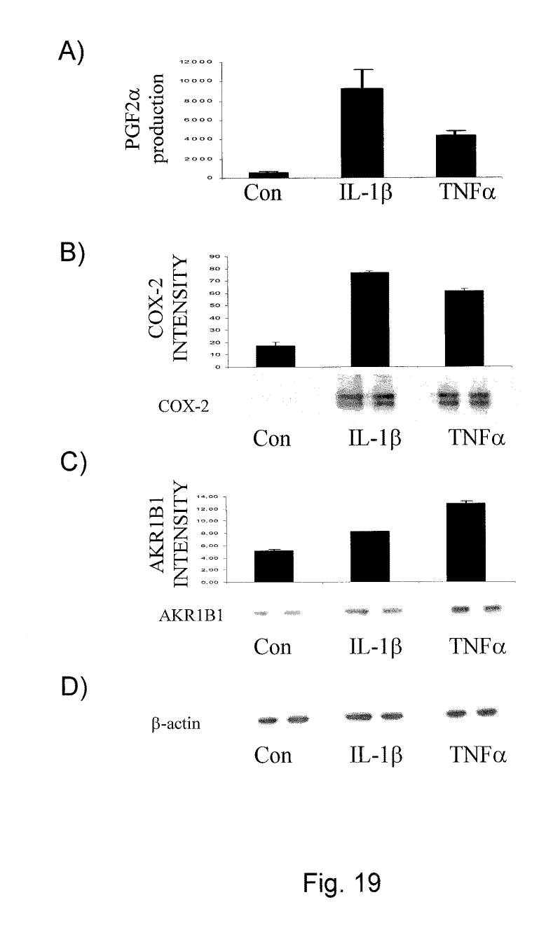

Low expression levels (basal) of AKR1B1 have been suggested to be involved in the protection against oxidative or electrophilic stresses (US 2006/0293265). In contrast, overexpression of AKR1B1 associated with COX is producing increased levels of PGF_{2α}, which leads to pain via ischemia as previously shown with menstrual pain. However, since glucose is a poor substrate of aldose reductase, the PGFS activity of AKR1B1 therefore predominates in the whole organism since AKR1B1 is ubiquitously expressed, despite a greater expression in skeletal muscle, cardiac muscle, kidney, ovary, testis, prostate and small intestine (Jin et al, Annu Rev Pharmacol Toxicol, 47; 263-92, 2007). Activation or overexpression of AKR1B1 is achieved in response to primary signals such as osmotic shock, reactive oxygen species (ROS), and other localized stress agents. Depending on the physiological and toxicological context, the beneficial or detrimental effects associated with the expression level of AKR1B1 are related to the synthesis level of PGF_{2α} from PGH₂. Our laboratory has clearly shown that, in human endometrium, cytokines such as IL-1β and TNF-α (inflammatory and apoptotic cytokines) both increased the expression levels of COX-2 and AKR1B1 simultaneously (FIG. 19), which resulted in greatly increased levels of PGF_{2α} involved in menstrual pain. In fact, both IL-1β and TNF-α stimulated AKR1B1 and COX-2 protein expression and PGF_{2α} production, thus showing co-regulation of AKR1B1 protein and PGF_{2α} production.

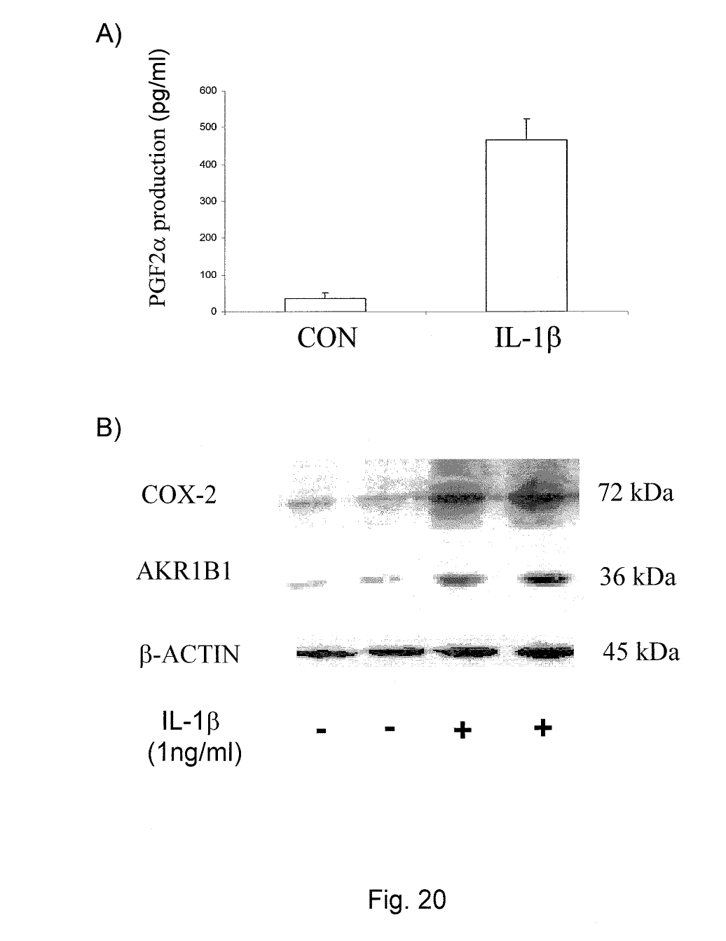

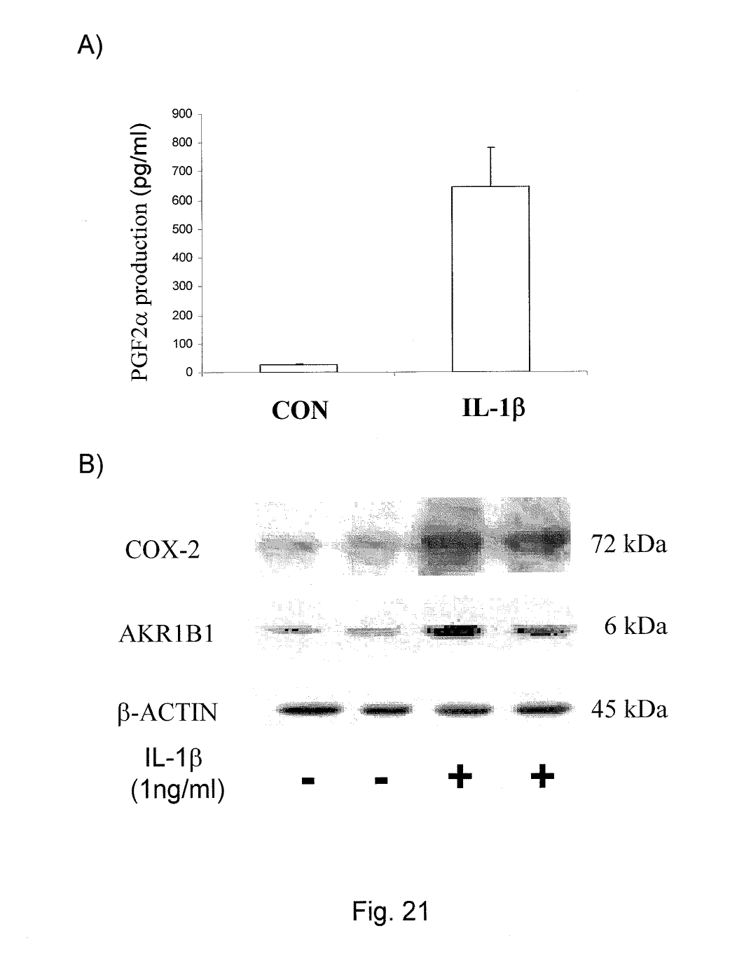

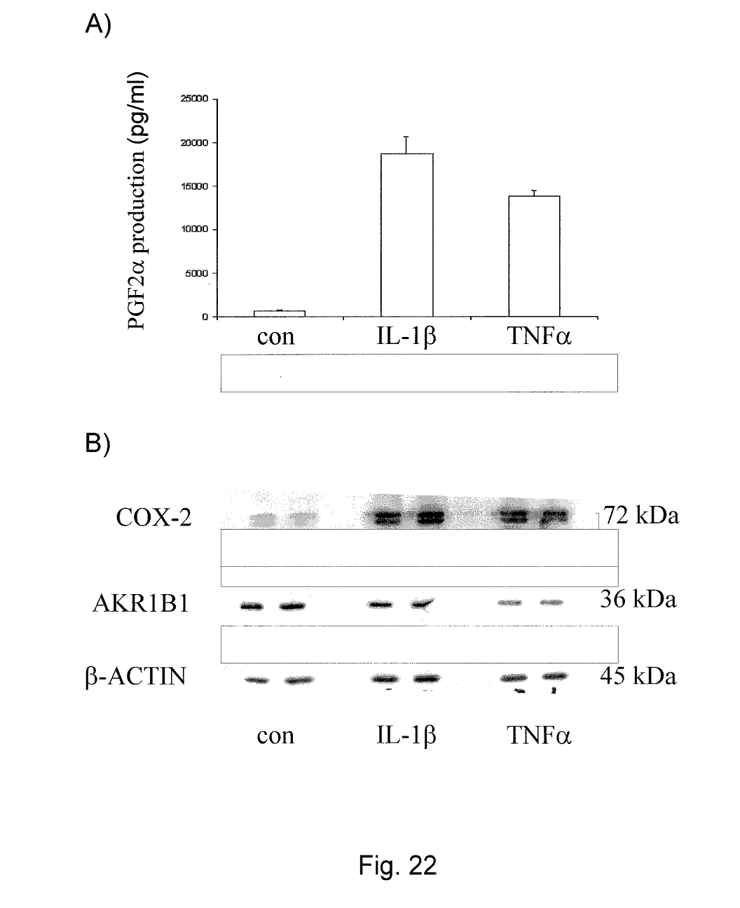

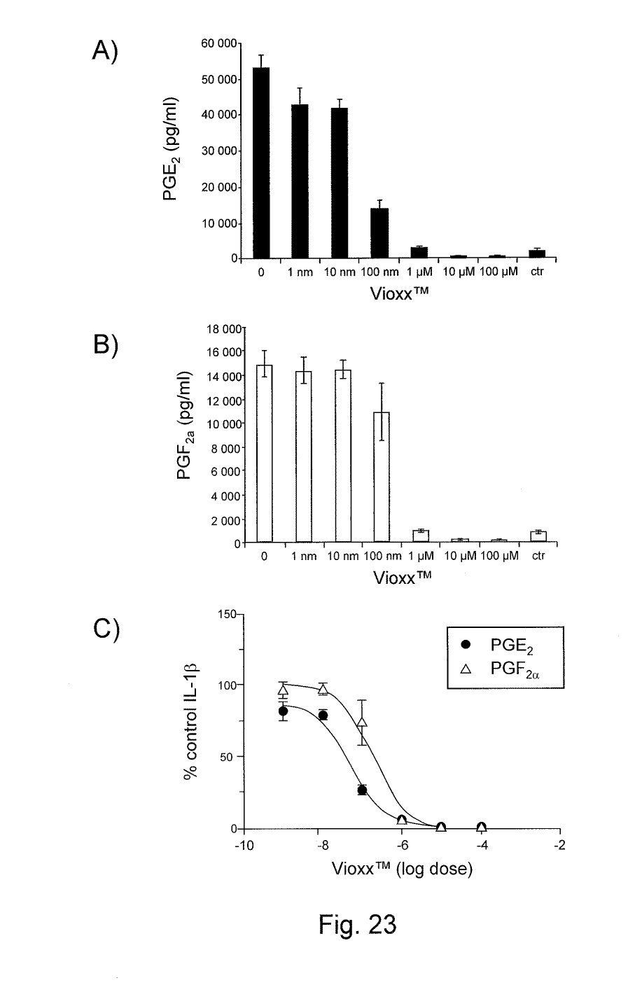

Further, we showed that IL-1β stimulated AKR1B1 and COX-2 protein expression, as well as PGF_{2α} production, in primary human umbilical artery smooth muscle cells (FIG. 20) and in primary human umbilical vein smooth muscle cells (FIG. 21), thus showing co-regulation of AKR1B1 protein and PGF_{2α} production in those systems. We also demonstrated that IL-1β and TNF-α stimulated COX-2 protein expression and PGF_{2α} production while endogenous AKR1B1 levels were already high in primary human umbilical vein endothelial cells (FIG. 22), thus showing the association between AKR1B1 protein and PGF_{2α} production in this system.

In addition, a recent study demonstrated that AKR1B1 inhibitors such as Sorbinil™, Tolrestat™ and Zopolrestat™ were capable to reduce the production levels of PGE₂ produced by macrophages treated with endotoxines or lipopolysaccharides (Ramana K. V. et al., 2006). In accordance with the present invention, this would result from a decrease in PGF_{2α} production by AKR1B1, which is disrupting the equilibrium between the different PGs. Furthermore, ROS like hydrogen peroxide are capable of increasing both COXs and PGF_{2α} expression levels in the endometrium, but to a lesser extent than cytokines.

In addition to demonstrating the PGFS activity of AKR1B1 and the regulation of its expression by IL-1β, we have previously characterized its gene promoter region. It showed an association between PGF_{2α} production and gene regulatory signals acting on osmotic response elements (ORE), antioxidant response elements (ARE) and AP-1 sites, all of which were previously shown to increase the expression level of AKR1B1 (Jin Y et al., Annu Rev Pharmacol Toxicol 47; 263-92. 2007).

Moreover, we investigated if the cytosolic nature of AKR1B1 allowed for its coupling with either COX-1 or COX-2, or with a pool of COX-2 distinct from the one used by mPGES-1 and which would remain available in the presence of a COX-2-specific inhibitor such as rofecoxib (Vioxx™). FIG. 27 shows that rofecoxib inhibits the production of PGE₂ by endometrial epithelial HIEEC cells stimulated with IL-1β ten times more efficiently than the production of PGF_{2α} by the same cell type, which expresses both COX-1 and COX-2. At 10 μM rofecoxib thus induces a strong alteration of the PGF_{2α}/PGE₂ ratio in favor of PGF_{2α}, an effect opposed to that observed with naproxen. Note that the response observed in endometrial cell lines are characteristic for each inhibitor tested and that the effect on the PGF_{2α}/PGE₂ ratio reflects the relative cardiovascular safety of rofecoxib vs naproxen.

Therefore, under conditions of aberrant overexpression of AKR1B1, the use of NSAIDs, especially COX-2-specific inhibitors, to treat pain or inflammation may prevent the compensatory release of relaxing PGE₂, thus leading to ischemic responses like those observed with Vioxx™ and Bextra™. Indeed, since PGs possess a compensation mechanism based on the expression of the different PG isotypes and receptors having antagonistic effects, undesirable side-effects of NSAIDs, COX-2-specific inhibitors and AKR1B1 inhibitors could originate from a perturbation in the equilibrium of the various PGs produced.

Consequently, the PGFS activity of AKR1B1 is a primary activity of this enzyme and represents a therapeutic target for the development and validation of modulators of its expression or activity. Moreover, FP receptor blockers and PGF_{2α} agonists analogs acting on FP receptors are to be considered as efficient tools for controlling the aberrant PGFS activity of AKR1B1, or for compensating for the lack of PGFS activity of AKR1B1 that could result, for example, from a congenital disorder or from a pharmacological inhibition.

### Example 4

### Evaluation of the Safety and/or the Risk Related to the Use of a COX-2 Specific Inhibitor by a Subject

PGFM is a stable metabolite of PGF_{2α} cleared in urine and that could be used in a diagnosis test to evaluate the metabolic status of any subjects if expressed relatively to PGEM levels. Normal subjects with normal PGF_{2α} and PGE₂ levels have an equilibrated PGFM/PGEM ratio with low absolute values. Subjects with insulin resistance should also have an equilibrated ratio, but with higher absolute levels of both metabolites. Subjects at risk of cardiovascular events would however have a higher PGFM/PGEM ratio, reflective of a higher concentration of PGFM relative to the concentration of PGEM.

Thus, the same ratio can be used as a safety measure before and during use of COX-2-specific inhibitors in a subject having insulin resistance, type 2 diabetes, or any other disorder or symptom motivating the administration of a COX-2-specific inhibitor. For example, a ratio PGF/PGE is used in order to monitor the safety of prescribing a COX-2-specific inhibitor to a subject having insulin resistance or type 2 diabetes. The determination of the PGF/PGE ratio is performed by measuring the concentration of PGF variants and the concentration of PGE variants in a biological sample, such as blood, urine or tissues, with an immunoassay such as a radioimmunoassay or an ELISA.

An ELISA test kit is developed with goat antimouse IgG antibody-coated microtiter plate wells. Controls and samples are introduced into the wells, and PGFM and/or PGEM tracers are added for example, in the case where the ratio to be observed is a PGFM/PGEM ratio. It will be understood that an ELISA kit can be developed using PGF₂₀ and PGE₂ tracers, or any other PGF₂₀ and PGE₂ variants, with the appropriate antibodies. The two tracers can be put together into a single well or in two separate wells, depending on the design of the ELISA test. Tracers can be conjugated with any kind of detection system, such as alkaline phosphatase or acetyl cholinesterase. The addition of a mouse monoclonal anti-PGFM and/or anti-PGEM (Cayman Chemical Company, MI USA) initiates the reaction. During incubation, there is a competition between the PGFM and/or PGEM present in the samples and the tracers for binding to the mouse anti-PGFM and/or anti-PGEM bound to the wells via the goat anti-mouse IgG antibody. Washing of the wells after the incubation period removes the unbound PGFM and/or PGEM, and addition of a substrate of the enzyme, such as p-nitrophenyl phosphate for alkaline phosphatase for example, allows for the plate to be optically read at a given wavelength, such as 405 nm. Addition of EDTA can be performed prior to reading to terminate the enzymatic reaction.

When the samples contain high levels of PGFM and/or PGEM, there is less tracers bound to the monoclonal antibodies, which results in lower optical density values. Lower levels of PGFM and/or PGEM do in turn produce higher optical density readings caused by the binding of a higher proportion of tracers to the monoclonal antibodies. The actual concentrations of PGFM and/or PGEM can therefore be calculated from the comparison of the optical densities of the samples with a reference curve established from the optical densities of the control wells having a known concentration of PGFM and/or PGEM.

If the measurement is performed in urine, urine samples are to be used in the test in order to normalize from urine dilution by obtaining the urinary creatinine values.

### Example 5

### Determination of the Predisposition of a Subject to a Metabolic Disorder or to a Cardiac Problem

Immunoassays as described in example 4 can be used for predicting the predisposition or risk of a subject to develop a cardiac problem, such as cardiac ischemia or heart failure, before or after the occurrence of a metabolic disorder, such as obesity, type 2 diabetes or insulin resistance. Cardiac problems as used herein are also intended to encompass myocardial infarction and its complication, such as congestive heart failure, myocardial rupture, arrhythmia, cardiogenic shock and pericarditis. Additional examples of metabolic disorders includes, in a non-limitative manner, disorders of carbohydrate metabolism, disorder of amino acid metabolism, disorder of organic acid metabolism, disorder of fatty acid oxidation and mitochondrial metabolism, disorder of porphyrin metabolism, disorder of purin or pyrimidine metabolism, disorder of steroid metabolism, disorder of mitochondrial function, disorder of peroxisomal function, and disorder of lysosomal storage Non-limitative examples of metabolic disorder complications includes diabetes, osteoporosis, menstrual disorders, neuropathy, retinopathy, and cataracts.

Controls to be used for such a determination are to be reflective of the various stages or severity levels for the tested metabolic disorder or cardiac problem, that is with control values for various types of type 2 diabetes for example being reflective of the severity of the predisposition.

We believe that some COX-2-specific inhibitors, such as Vioxx™, do not target COX-2 activity alone, but rather the biosynthetic complex formed by the association of COX-2 and PGE₂, making it highly efficient to block pain and inflammation, but also more prone to induce an aberrant PGF/PGE ratio. We therefore propose that the different prevalence of cardiovascular complications amongst NSAIDs and COX-2-specific inhibitors users depends on the relative ability of AKR1B1 to generate PGF variants in the presence of those drugs. This can be determined in vitro by comparing various PGF/PGE ratios obtained in presence of various doses of COX-2-specific inhibitors, and monitored in vivo by measuring PGFM and PGEM in urine and/or blood of the subjects. An immediate treatment in the case of a highly unbalanced PGF_{2α}/PGE₂ ratio for example could be the administration of a PGF_{2α} receptor antagonist.

### Example 6

### Modulation of the PGFS Activity of AKR1B1 for the Prevention of Cardio-Vascular Problems in Subjects Having Insulin Resistance or Type 2 Diabetes

PGs-related compounds such as TXA₂ and PGI₂, are chemically unstable and strictly act locally at their site of biosynthesis. However, PGF_{2α} and PGE₂ have the chemical stability to allow action on cells and tissues adjacent to the site of production through a paracrine action limited only by the PG transport system that we have described in the bovine and human uterus. None of the PGs can exert a systemic response because after entering general circulation they are enzymatically degraded in the lungs.

COX-1 is a constitutive enzyme that reacts instantly to elevated concentrations of AA, transforming it into PGH₂, which is next converted into PGF_{2α} by AKR1C3 or AKR1B1 as needed. COX-2 is an inducible enzyme, reacting to lower concentrations of AA than COX-1, and transforming it into PGH₂, which, most of the time, will be converted into PGE₂ by mPGES-1. PGE₂ can be converted into PGF_{2α} by a 9 KPGR, in order to maintain a PGF_{2α}/PGE₂ ratio suitable for an equilibrium of the opposed effects of those two PGs (Farina, M G et all 2006 POLM 79; 260-270). During the menstrual cycle, if this ratio switches in favor of PGE₂, the subject will present abundant bleeding. By opposition, if the ratio switches in favor of PGF_{2α}, it will induce myometrial and vascular contractions, which can lead to myometrial ischemia and menstruation-related pain.

We examined the effect of a knock-down of the genes encoding the two terminal PG synthases mPGES-1 and AKR1B1 on PGs production in cells. A knock-down of mPGES-1 induced a decrease only in the synthesis PGE₂, as expected, but surprisingly, the knock-down of AKR1B1 induced a decrease in the synthesis of both PGF_{2α} and PGE₂. Therefore, it appeared that when AKR1B1 was blocked, mPGES-1 activity but not expression was also blocked. Conversely, as verified by a knock-in of the AKR1B1 gene, an increase in AKR1B1 activity also induced an increase in mPGES-1 activity, leading to PGE₂ synthesis. Therefore, it seems that there is a cross talk between the biosynthetic enzymes leading to PGE₂ and PGF_{2α} thus ensuring the balance between the two. Increased PGF_{2α} resulting from excess AKR1B1 activity in response to osmotic stress, insulin resistance or else would then be compensated by increase mPGES-1 and PGE₂. However, if this mechanism is blocked such as in presence of Vioxx™, excess PGF_{2α} will eventually build up and combined vasoconstriction and increased left ventricle contraction generate ischemic responses and heart failure.

Glucose, along with ROS, induces AKR1B1 expression, which will convert glucose into sorbitol. Insulin also induces the expression of AKR1B1 gene through p38MAPK and PI3K. However, the conversion of glucose into sorbitol only occurs at high concentrations of glucose, such as in diabetic subjects. During normal glycemia, AKR1B1 does not convert glucose, but it will still continue to exert its other activities, such as PGFS and detoxification of peroxidized lipids. In subjects presenting insulin resistance, the glucose transport is affected, which results in a higher production of insulin for preserving the glycemia at a level close to normal. But because only the PI3K component of the insulin receptor is desensitized, the higher concentrations of insulin will likely induce an increase in the expression rate of insulin-responsive genes regulated by the p38MAPK transduction system, including the AKR1B1 gene (Kang E S et al Free radical Biology and Medicine 43: 535-545 2007).

AKR1B1 possesses two enzymatic pockets one rigid and one adaptative (Steuber et al., J Mol Biol 369(1); 186-97, 2007). Therefore, it is theoretically possible to interfere or regulate its activity without binding on the active rigid site, or compete directly with other substrates. This may explain how glucose exhibiting a molecular structure distinct from PGH₂ can both be metabolized by AKR1B1 and interfere with its PGFS activity.

Aspirin™ blocks the expression of the AKR1B1 gene, but COX-2-specific inhibitors, such as Vioxx™, do not influence AKR1B1 gene expression. However, COX-2-specific inhibitors are likely to block preferentially the formation of PGE₂, because mPGES-1, the most important inducible PGES, is strictly associated with COX-2 to produce PGE₂. However, as previously mentioned, in cases of diabetes, oxidative stress and insulin resistance, the expression of AKR1B1 is increased. If any subject described previously experiences chronic pain symptoms, knowing that COX-2-specific inhibitors are amongst the most efficient anti-inflammatory and analgesic drug, they will be prescribed with COX-2-specific inhibitor or NSAIDs. However, since AKR1B1 expression is increased, preferred blocking of PGE₂ synthesis will switch the equilibrium of PGs toward a high concentration of PGF_{2c}, compared with a much lower concentration of PGE₂ thus favoring ischemic responses.

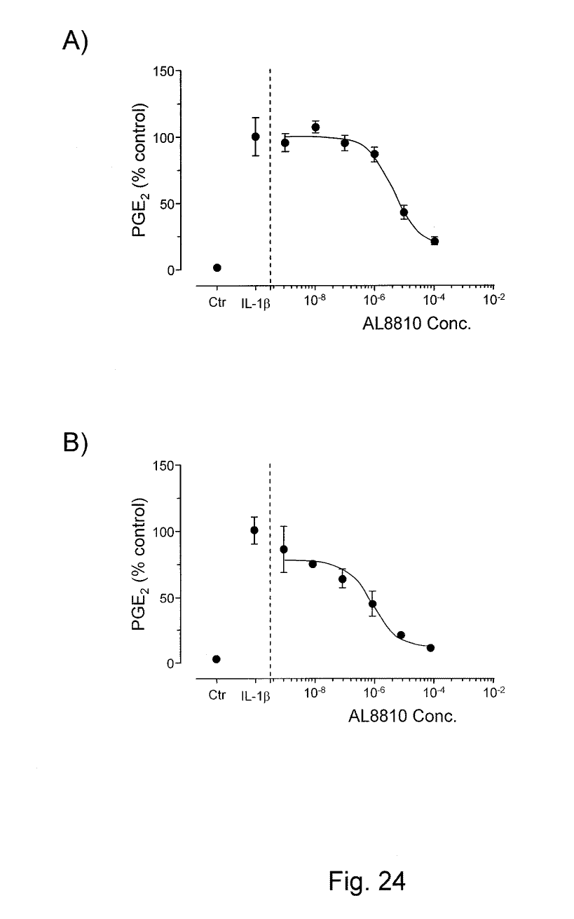

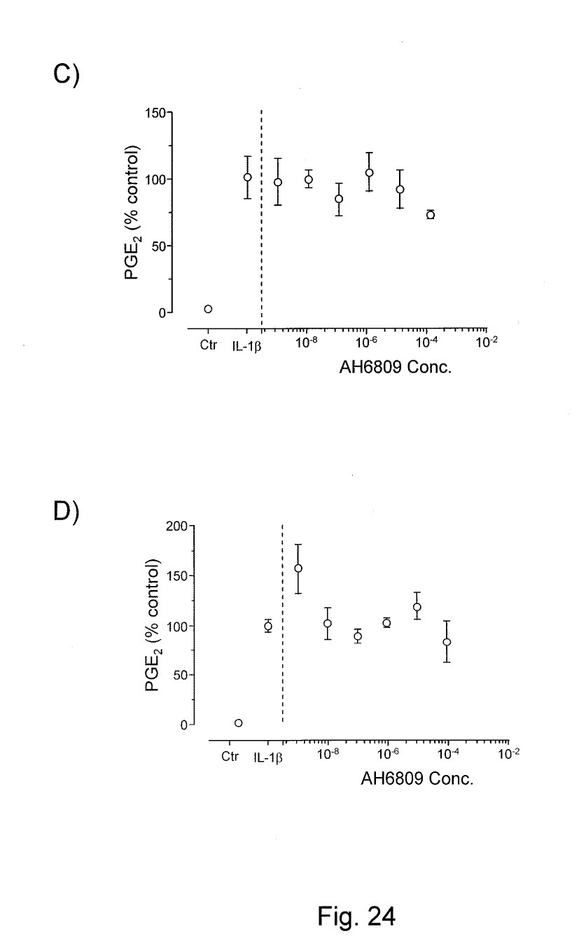

Because of the promiscuity between mPGES-1 and COX-2, COX-2-specific inhibitors such as Vioxx™ are particularly efficient in blocking PGE₂, they often represent the preferred option to block pain and inflammation. However, they are also more prone to induce aberrant PGF/PGE ratios. We claim that the different prevalence of cardiovascular complications amongst NSAIDs and COX-2-specific inhibitors depends on the relative ability of AKR1B1 to generate PGF_{2α} in their presence. This can be determined in vitro by comparing the PGF/PGE ratios in presence of various doses of COX-2-specific inhibitors and monitored in vivo by measurement of PGFM and PGEM in urine and blood (FIG. 13). An immediate treatment in case of a high PGF_{2α}/PGE₂ ratio for example would be to administer an FP receptor antagonist to block the ischemic response. FIG. 24 shows the comparative effects of an inhibitor of FP receptor (AL8810) and an inhibitor of EP receptor (AH6809) on the production of PGE₂ by IL-1β-stimulated endometrial epithelial and stromal cells. Inhibition of FP receptors, but not of EP receptors, induced a reduction in PGE₂ production, thus suggesting that PGF_{2α} exerts an upregulation of PGE₂ production in both endometrial cell lines, as illustrated in FIG. 7.

Further, we show that the PGFS activity of AKR1B1 can be used as a therapeutic target to decrease the risks of COX-2-specific inhibitor-associated cardio-vascular disorders in subjects exhibiting aberrant overexpresssion of AKR1B1, such as in subject having insulin resistance or type 2 diabetes for example.

Such compounds for targeting AKR1B1 as a therapeutic target can be identified by traditional methods of drug screening, mainly by exposing a cell to a COX inhibitor and studying the effect of those compounds on the AKR1B1 expression or activity, on the PGF_{2α} expression or activity, or on the PGF/PGE ratio in the cell. The exposition of the cell to the COX inhibitor can be reflective of a short term exposure (from 3 to 6 hours exposition) or of a long term exposure (from 2 to 7 days exposition). While such testing can be performed in vitro on human endometrial epithelial cells, human endometrial stromal cells, adipocytes, endothelial cells, human umbilical vein endothelial cells, kidney cells, HEK293 cells, smooth muscle cells, myoblasts, heart cells and cardiomyocytes, in vivo testing can also be performed on traditional animal models such as mouse or rats. Preferably, all cells are human cells, and the animal models for in vivo tests are transgenic animals expressing or overexpressing human AKR1B1. Cell lines such as human endometrial stromal cell line HIESC-2 (IDAC number 301008-04) and human endometrial epithelial cell line HIEEC-22 (IDAC number 301008-05) can also be used for in vitro tests.

Use of a PGF/PGE ratio for the identification of a compound alleviating a COX inhibitor-associated side-effect, wherein said compound induces a decrease in the value of PGF/PGE ratio in a cell treated with said COX inhibitor.

### Example 7

### Relationship Between Fatty Acids and AKR1B1

Omega-3 and omega-6 fatty acids have been greatly publicized over the last few years. Since PGs possess a 20 carbon atoms structure derived from fatty acids, they can easily be synthesized from omega-3 and -6 fatty acids having 20 carbons, such as from the omega-3 fatty acid eicosapentaenoic acid (EPA), and from the omega-6 fatty acids dihomo-gamma-linolenic acid (DGLA) and arachidonic acid (AA). For example, under the action of COX, DGLA can be converted into PGH₁, AA into PGH₂ and EPA into PGH₃, which, under the effect of the PGFS action of AKR1B1, can respectively be converted into series 1, 2 and 3 prostaglandin F variants, namely PGF_{1α}, PGF_{2α} and PGF_{3α}.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. The method of claim 55, wherein said modulating decreases the PGFS activity in the subject and the modulator is an inhibitor of the PGF synthase activity of AKR1B1 (EC 1.1.1.21)

2. The method of claim 57, wherein said modulating decreases the level of PGF_{2α} in the subject, and the modulator is an inhibitor of the PGF synthase activity of AKR1B1 (EC 1.1.1.21).

3. The method of claim 58, wherein the altered PGF_{2α} levels or activity are increased in the subject and the modulator is an inhibitor of the PGF synthase activity of AKR1B1 (EC 1.1.1.21).

4. The method of claim 3, wherein said condition associated with an increase of PGF_{2α} levels or activity in a subject is selected from the group consisting of metabolic disorders, metabolic disorder complications, cardiac ischemia, cerebral ischemia, bronchial constriction, menstrual pain, renal dysfunction and premature labor.

5. The method of claim 1, wherein said inhibitor is selected from the group consisting of: an inhibitor of AKR1B1 synthesis, an inhibitor of AKR1B1 translation, an inhibitor of AKR1B1 post-translational modification, a regulator of AKR1B1 transit within the cytoplasm, and an activator of AKR1B1 degradation.

6. The method of claim 5, wherein said AKR1B1 inhibitor is selected from the group consisting of an AKR1B1 siRNA and an AKR1B1 antibody.

7. The method of claim 6 further comprising the step of administering to said subject at least one of a COX inhibitor, an FP receptor blocker, an EP1 receptor blocker, an EP3 receptor blocker, and a PGF_{2α} antagonist.

8. The method of claim 7, wherein said COX inhibitor is a COX-2-specific inhibitor.

9. The method of claim 1, wherein said subject is a human subject.

10. The method of claim 55, wherein said modulating increases the PGFS activity in the subject and the modulator is an activator of the PGF synthase activity of AKR1B1.

11. The method of claim 57, wherein said modulating increases the level of PGF_{2α} in the subject and the modulator is an activator of the PGF synthase activity of AKR1B1.

12. The method of claim 58, wherein the altered PGF_{2α} levels or activity are decreased in the subject and the modulator is an activator of the PGF synthase activity of AKR1B1.

13. The method of claim 12, wherein said condition is selected from the group consisting of hyperglycemia, inflammation and impaired renal function.

14. The method of claim 10, wherein said activator is selected from the group consisting of an activator of AKR1B1 synthesis, an activator of AKR1B1 translation, an activator of AKR1B1 binding, an inhibitor of AKR1B1 degradation, an AKR1B1 gene and an AKR1B1 protein.

15. The method of claim 14, wherein said AKR1B1 activator is selected from the group consisting of a nucleic acid encoding at least the PGFS activity portion of AKR1B1 and a polypeptide having at least the PGFS activity of AKR1B1.

16. The method of claim 10, further comprising the step of administering to said subject at least one of a COX activator, an FP receptor activator, an EP1 receptor activator, an EP3 receptor activator, and a PGF_{2α} agonist.

17. The method of claim 16, wherein said COX activator is a COX-2-specific activator.

18. The method of claim 10, wherein said subject is a human subject.

19-54. (canceled)

55. A method for modulating PGFS activity in a subject, said method comprising the step of administering a modulator of the PGF synthase activity of AKR1B1 (EC 1.1.1.21) to said subject.

56. The method of claim 1, wherein the subject suffers from an overproduction of PGF_{2α}.

57. A method for modulating the level of PGF_{2α} in a subject, said method comprising the step of administering a modulator of the PGF synthase activity of AKR1B1 to said subject.

58. A method for treating or preventing a condition associated with altered PGF_{2α} levels or activity in a subject, said method comprising the step of administering a modulator of PGF synthase activity of AKR1B1 (EC 1.1.1.21) to said subject.


SEQUENCE LISTING (6 sequences)

SEQ ID NO: 1 - DNA, 23 bp, Artificial Sequence
  Synthetic Oligonucleotide - AKR1B1 forward primer
gatgagtcgggcaatgtggttcc

SEQ ID NO: 2 - DNA, 23 bp, Artificial Sequence
  Synthetic Oligonucleotide - AKR1B1 reverse primer
cttggctgcgatcgccttgatcc

SEQ ID NO: 3 - DNA, 23 bp, Artificial Sequence
  Synthetic Oligonucleotide - AKR1C3 forward primer
ctaaagccaggtgaggaactttc

SEQ ID NO: 4 - DNA, 23 bp, Artificial Sequence
  Synthetic Oligonucleotide - AKR1C3 reverse primer
ctatcactgttaaaatagtggag

SEQ ID NO: 5 - DNA, 41 bp, Artificial Sequence
  Synthetic Oligonucleotide - AKR1B1 siRNA forward primer
aaattgttgagcaggagacggctatagtgagtcgtattacc

SEQ ID NO: 6 - DNA, 41 bp, Artificial Sequence
  Synthetic Oligonucleotide - AKR1B1 siRNA reverse primer
aagccgtctcctgctcaacaactatagtgagtcgtattacc